SEQUENCE LISTING (24 sequences)

SEQ ID NO: 1 - protein, 10 aa, synthetic construct
GFSLDYYAIG

SEQ ID NO: 2 - protein, 10 aa, synthetic construct
CISSSGGSTD

SEQ ID NO: 3 - protein, 14 aa, synthetic construct
GVSGSVCPPLIGAY

SEQ ID NO: 4 - protein, 121 aa, synthetic construct
QVQLVESGGGLVQAGGSLRLSCAASGFSLDYYAIGWFRQAPGKEREEVSCISSSGGSTDYADSVKGRFTISRAYAKVYLQMNSLKPEDTAVYYCAAGVSGSVCPPLIGAYWGQGTQVTVSS

SEQ ID NO: 5 - protein, 10 aa, synthetic construct
GFPLDYYAIG

SEQ ID NO: 6 - protein, 9 aa, synthetic construct
CIISGGSTK

SEQ ID NO: 7 - protein, 16 aa, synthetic construct
SWSCDLFPRDTYGWDA

SEQ ID NO: 8 - protein, 124 aa, synthetic construct
EVQLVESGGGLVQPEGSLRLSCAASGFPLDYYAIGWFRQAPGKEREGVSCIISGGSTKYSASVKGRFTISRDSANNTVYLQMNSLKPEDTAVYFCAVSWSCDLFPRDTYGWDAWGQGTLVTVSS

SEQ ID NO: 9 - protein, 350 aa, synthetic construct
QVQLVESGGGLVQAGGSLRLSCAASGFSLDYYAIGWFRQAPGKEREEVSCISSSGGSTDYADSVKGRFTISRAYAKVYLQMNSLKPEDTAVYYCAAGVSGSVCPPLIGAYWGQGTQVTVSSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 353 aa, synthetic construct
EVQLVESGGGLVQPEGSLRLSCAASGFPLDYYAIGWFRQAPGKEREGVSCIISGGSTKYSASVKGRFTISRDSANNTVYLQMNSLKPEDTAVYFCAVSWSCDLFPRDTYGWDAWGQGTLVTVSSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 121 aa, synthetic construct
QVQLVESGGGLVQPGGSLRLSCAASGFSLDYYAIGWFRQAPGKEREEVSCISSSGGSTDYADSVKGRFTISRAYAKVYLQMNSLKPEDTAVYYCAAGVSGSVCPPLIGAYWGQGTLVTVSS

SEQ ID NO: 12 - protein, 350 aa, synthetic construct
QVQLVESGGGLVQPGGSLRLSCAASGFSLDYYAIGWFRQAPGKEREEVSCISSSGGSTDYADSVKGRFTISRAYAKVYLQMNSLKPEDTAVYYCAAGVSGSVCPPLIGAYWGQGTLVTVSSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 121 aa, synthetic construct
QVQLVESGGGLVQAGGSLRLSCAASGFSLDYYAIGWFRQAPGKEREEVSCISSSGGSTDYADSVKGRFTISRAYAKVYLQMNSLRAEDTAVYYCAAGVSGSVCPPLIGAYWGQGTLVTVSS

SEQ ID NO: 14 - protein, 350 aa, synthetic construct
QVQLVESGGGLVQAGGSLRLSCAASGFSLDYYAIGWFRQAPGKEREEVSCISSSGGSTDYADSVKGRFTISRAYAKVYLQMNSLRAEDTAVYYCAAGVSGSVCPPLIGAYWGQGTLVTVSSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 15 - protein, 121 aa, synthetic construct
QVQLVESGGGLVQAGGSLRLSCAASGFSLDYYAIGWFRQAPGKGLEEVSCISSSGGSTDYADSVKGRFTISRAYAKVYLQMNSLRAEDTAVYYCAAGVSGSVCPPLIGAYWGQGTLVTVSS

SEQ ID NO: 16 - protein, 350 aa, synthetic construct
QVQLVESGGGLVQAGGSLRLSCAASGFSLDYYAIGWFRQAPGKGLEEVSCISSSGGSTDYADSVKGRFTISRAYAKVYLQMNSLRAEDTAVYYCAAGVSGSVCPPLIGAYWGQGTLVTVSSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 17 - protein, 124 aa, synthetic construct
EVQLVESGGGLVQPEGSLRLSCAASGFPLDYYAIGWFRQAPGKEREGVSCIISGGSTKYSASVKGRFTISRDSAKNTVYLQMNSLRAEDTAVYFCAVSWSCDLFPRDTYGWDAWGQGTLVTVSS

SEQ ID NO: 18 - protein, 353 aa, synthetic construct
EVQLVESGGGLVQPEGSLRLSCAASGFPLDYYAIGWFRQAPGKEREGVSCIISGGSTKYSASVKGRFTISRDSAKNTVYLQMNSLRAEDTAVYFCAVSWSCDLFPRDTYGWDAWGQGTLVTVSSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 19 - protein, 229 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 20 - protein, 10 aa, synthetic construct
RTDSNIHGMH

SEQ ID NO: 21 - protein, 9 aa, synthetic construct
MIFIDGNTI

SEQ ID NO: 22 - protein, 8 aa, synthetic construct
DSRGYGRA

SEQ ID NO: 23 - protein, 116 aa, synthetic construct
EVQLVESGGGLVQPGGSLRLSCAASRTDSNIHGMHWYRQAPGKGREWVGMIFIDGNTIVTDSVKGRFTISRDNAKNTLYLQMNTLRAEDTAVYYCAADSRGYGRAWGQGTTVTVSS

SEQ ID NO: 24 - protein, 348 aa, synthetic construct
EVQLVESGGGLVQPGGSLRLSCAASRTDSNIHGMHWYRQAPGKGREWVGMIFIDGNTIVTDSVKGRFTISRDNAKNTLYLQMNTLRAEDTAVYYCAADSRGYGRAWGQGTTVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2026/0028402 A1
(43) Pub. Date: Jan. 29, 2026

(54) ANTI-CD100 ANTIBODY AND USE THEREOF

(71) Applicant: SANYOU BIOPHARMACEUTICALS CO., LTD., Minhang District Shanghai (CN)

(72) Inventors: Shuangqi LI, Shanghai (CN); Guojun LANG, Shanghai (CN); Xintian YAN, Shanghai (CN); Yuhao HU, Shanghai (CN); Zhen ZHANG, Shanghai (CN); Liyan WANG, Shanghai (CN); Xiaopeng FANG, Shanghai (CN); Peifang XU, Shanghai (CN); Yangyang HAN, Shanghai (CN); Yuqi REN, Shanghai (CN); Qiangqiang DENG, Shanghai (CN); Xue GAO, Shanghai (CN)

(21) Appl. No.: 19/134,118

(22) PCT Filed: Oct. 31, 2023
(86) PCT No.: PCT/CN2023/128251

§ 371 Date: May 29, 2025

(30) Foreign Application Priority Data
Nov. 30, 2022 (CN) 202211519754.9

(51) Int. Cl. C07K 16/28; A61K 39/00; A61K 39/395; A61P 35/00

(52) U.S. Cl. CPC C07K 16/2803; A61K 39/39558; A61P 35/00; A61K 2039/507; C07K 2317/24; C07K 2317/52; C07K 2317/565; C07K 2317/569; C07K 2317/73; C07K 2317/76; C07K 2317/92

(57) ABSTRACT

The present invention relates to the field of biomedicines. Specifically, the present invention relates to an anti-CD100 antibody and a use thereof, and a pharmaceutical composition or pharmaceutical combination containing the antibody and a use thereof. The present invention further provides a nucleic acid molecule and expression vector for encoding the antibody and a method for producing the antibody.

### TECHNICAL FIELD The present invention relates to the field of biomedicines. Specifically, the present invention relates to anti-CD100 antibodies and uses thereof.

### BACKGROUND

CD100, also known as semaphorin 4D (SEMA4D), is a transmembrane protein belonging to the semaphorin gene family. CD100 is expressed on the cell surface in the form of a homodimer and can be released from the cell surface through proteolysis, resulting in an active soluble form of CD100. CD100 is predominantly and strongly expressed in human lymphoid tissues, skeletal muscle, and the brain (at lower levels). Its biological activity is mainly characterized in the immune system: for example, as a receptor, CD100 can enhance T cell proliferation; as a ligand, it can promote the aggregation and survival of B cells and the activation and maturation of antigen-presenting cells (dendritic cells and macrophages). Through the high affinity receptor Plexin-B1, CD100 can inhibit the migration of monocytes and B cells.

CD100 is widely expressed in many human tumors, and its expression is associated with human aggressive diseases. In preclinical tumor microenvironment, inflammatory cells and tumor cells express CD100 to regulate the infiltration, spatial distribution and activity of myeloid and lymphoid cells. CD100 binds to Plexin receptors located on myeloid cells in the tumor microenvironment. When the CD100 protein is blocked, the CD100 barrier can be eliminated. Once the barrier is breached, inflammatory dendritic cells and pro-inflammatory antigen-presenting cells migrate and infiltrate into the tumor. In preclinical cancer animal models, blocking CD100 with antibodies can delay tumor growth and promote durable tumor rejection.

Several antibodies against CD100 have been developed in the prior art, such as Pepinemab (Vaccinex, Inc.). However, there remains a need in the art for anti-CD100 antibodies that are capable of specifically binding to CD100 and blocking the binding of CD100 with its Plexin receptor. Nanobodies have a relatively small relative molecular weight, and have the advantages of ease of humanization, high affinity, low immunogenicity and the like. It is desirable in the art to develop anti-CD100 nanobodies that may specifically bind to CD100 and block the binding of CD100 with its Plexin receptor.

### SUMMARY

In an aspect, the present invention provides an antibody or an antigen-binding fragment thereof against CD100, comprising an immunoglobulin single variable domain, wherein the single variable domain comprises three CDR sequences in the amino acid sequence of the single variable domain as set forth in SEQ ID NO: 4 or a variant thereof; or the single variable domain comprises three CDR sequences in the amino acid sequence of the single variable domain as set forth in SEQ ID NO: 8 or a variant thereof; wherein the variant differs from the CDR sequence from which it is derived by addition, deletion or substitution of no more than 2 amino acids.

In an embodiment, the single variable domain comprises: (a) a CDR1 sequence as set forth in SEQ ID NO: 1; a CDR2 sequence as set forth in SEQ ID NO: 2; and a CDR3 sequence as set forth in SEQ ID NO: 3; or (b) a CDR1 sequence as set forth in SEQ ID NO: 5; a CDR2 sequence as set forth in SEQ ID NO: 6; and a CDR3 sequence as set forth in SEQ ID NO: 7.

In an embodiment, the antibody or antigen-binding fragment thereof comprises an immunoglobulin single variable domain, wherein the single variable domain comprises an amino acid sequence as set forth in SEQ ID NO: 4 or an amino acid sequence having at least 85%, at least 90%, at least 95% or higher sequence identity to SEQ ID NO: 4.

In an embodiment, the antibody or antigen-binding fragment thereof comprises an immunoglobulin single variable domain, wherein the single variable domain comprises an amino acid sequence as set forth in SEQ ID NO: 8 or an amino acid sequence having at least 85%, at least 90%, at least 95% or higher sequence identity to SEQ ID NO: 8.

In an embodiment, the antibody or antigen-binding fragment thereof comprises an immunoglobulin single variable domain, wherein the immunoglobulin single variable domain is fused to an additional molecule.

In a preferred embodiment, the additional molecule is an Fc domain of an immunoglobulin.

In a preferred embodiment, the additional molecule is an Fc domain of an immunoglobulin G4 (IgG4SP).

In an embodiment, the antibody or antigen-binding fragment thereof comprises an amino acid sequence as set forth in SEQ ID NO: 9 or an amino acid sequence having at least 85%, at 25 least 90%, at least 95% or higher sequence identity to SEQ ID NO: 9.

In an embodiment, the antibody or antigen-binding fragment thereof comprises an amino acid sequence as set forth in SEQ ID NO: 10 or an amino acid sequence having at least 85%, at least 90%, at least 95% or higher sequence identity to SEQ ID NO: 10.

In an embodiment, the antibody or antigen-binding fragment thereof is a humanized antibody or an antigen-binding fragment thereof.

In an embodiment, the humanized antibody or antigen-binding fragment thereof comprises an immunoglobulin single variable domain, wherein the single variable domain comprises an amino acid sequence as set forth in SEQ ID NO: 11, 13 or 15 or an amino acid sequence having at least 85%, at least 90%, at least 95% or higher sequence identity to SEQ ID NO: 11, 13 and 15.

In an embodiment, the humanized antibody or antigen-binding fragment thereof comprises an immunoglobulin single variable domain, wherein the single variable domain comprises an amino acid sequence as set forth in SEQ ID NO: 17 or an amino acid sequence having at least 85%, at least 90%, at least 95% or higher sequence identity to SEQ ID NO: 17.

In an embodiment, the humanized antibody or antigen-binding fragment thereof comprises an immunoglobulin single variable domain, wherein the immunoglobulin single variable domain is fused to an additional molecule.

In a preferred embodiment, the additional molecule is an Fc domain of an immunoglobulin.

In a preferred embodiment, the additional molecule is an Fc domain of an immunoglobulin G4 (IgG4SP).

In an embodiment, the humanized antibody or antigen-binding fragment thereof comprises an amino acid sequence as set forth in SEQ ID NO: 12, 14 or 16 or an amino acid sequence having at least 85%, at least 90%, at least 95% or higher sequence identity to SEQ ID NO: 12, 14 or 16.

In an embodiment, the humanized antibody or antigen-binding fragment thereof comprises the amino acid sequence as set forth in SEQ ID NO: 18 or an amino acid sequence having at least 85%, at least 90%, at least 95% or higher sequence identity to SEQ ID NO: 18.

In another aspect, the present invention further provides a pharmaceutical composition comprising the anti-CD100 antibody or antigen-binding fragment thereof of the present invention and a pharmaceutically acceptable carrier.

In another aspect, the present invention further provides a pharmaceutical combination comprising the anti-CD100 antibody or antigen-binding fragment thereof of the present invention and an anti-PD-L1 antibody or an antigen-binding fragment thereof.

In an embodiment, the anti-PD-L1 antibody or antigen-binding fragment thereof specifically recognizes and binds to PD-L1, wherein the PD-L1 antibody or antigen-binding fragment thereof comprises an immunoglobulin single variable domain.

In a preferred embodiment, the immunoglobulin single variable domain comprises a CDR1 sequence as set forth in SEQ ID NO: 20, a CDR2 sequence as set forth in SEQ ID NO: 21, and a CDR3 sequence as set forth in SEQ ID NO: 22.

In a preferred embodiment, the immunoglobulin single variable domain comprises: 1) an amino acid sequence as set forth in SEQ ID NO: 23; or 2) an amino acid sequence having at least 85%, at least 90%, at least 95% or higher sequence identity to SEQ ID NO: 23.

In an embodiment, the PD-L1 antibody or antigen-binding fragment thereof further comprises an Fc fragment of a human IgG1.

In a preferred embodiment, the PD-L1 antibody or antigen-binding fragment thereof comprises an amino acid sequence as set forth in SEQ ID NO: 24 or an amino acid sequence having at least 85%, at least 90%, at least 95% or higher sequence identity to SEQ ID NO: 24.

In an embodiment, the pharmaceutical combination is a pharmaceutical composition or a kit.

In another aspect, the present invention also provides use of the CD100 antibody or antigen-binding fragment thereof, the pharmaceutical composition, or the pharmaceutical combination of the present invention for the manufacture of a medicament for treating a cancer.

In a preferred embodiment, the cancer is a hematological tumor or a solid tumor.

In another aspect, the present invention further provides an isolated nucleic acid molecule encoding any one of the CD100 antibody or antigen-binding fragment thereof of the present invention.

In another aspect, the present invention further provides an expression vector comprising the nucleic acid molecule of the present invention.

In another aspect, the present invention further provides a host cell comprising the nucleic acid molecule or the expression vector of the present invention.

In another aspect, the present invention further provides a method for producing the CD100 antibody or antigen-binding fragment thereof of the present invention, comprising:
a) culturing the host cell of the present invention under suitable conditions to express the CD100 antibody or antigen-binding fragment thereof according to the present invention; and
b) isolating the antibody or antigen-binding fragment thereof from the host cell or a culture thereof.

### BRIEF DESCRIPTION OF THE DRAWINGS

FIG. 1 shows the ELISA detection results of Pepinemab antibody.

FIG. 2 shows the ELISA detection results of antigens from different species.

FIG. 3 shows the FACS detection results of overexpression cell lines from different species. FIGS. 4A-4D show the FACS detection results of Plexin receptor overexpression cell lines from different species. FIG. 4A shows the detection results of HuPlexin-B1-HEK293 overexpression cell lines. FIG. 4B shows the detection results of MusPlexin-B1-HEK293 overexpression cell lines. FIG. 4C shows the detection results of CynoPlexin-B1-HEK293 overexpression cell lines. FIG. 4D shows the detection results of HuPlexin-B2-HEK293 overexpression cell lines.

FIGS. 5A-5F show the ELISA detection results of the binding activity of the candidate antibodies to antigen proteins from different species. FIG. 5A shows the results of the binding activity of A9 to the HuCD100-His antigen protein. FIG. 5B shows the results of the binding activity of A9 to the MusCD100-His antigen protein. FIG. 5C shows the results of the binding activity of A9 to the CynoCD100-His antigen protein. FIG. 5D shows the results of the binding activity of A31 to the HuCD100-His antigen protein. FIG. 5E shows the results of the binding activity of A31 to the MusCD100-His antigen protein. FIG. 5F shows the results of the binding activity of A31 to the CynoCD100-His antigen protein.

FIGS. 6A-6C show the FACS detection results of the binding activity of the candidate antibodies to overexpression cell lines from different species. FIG. 6A shows the results of the binding activity of the candidate antibodies to the HuCD100-HEK293 overexpression cell line. FIG. 6B shows the results of the binding activity of the candidate antibodies to the MusCD100-HEK293 overexpression cell line. FIG. 6C shows the results of the binding activity of the candidate antibodies to the CynoCD100-HEK293 overexpression cell line.

FIGS. 7A and 7B show the FACS detection results of the binding activity of the candidate antibodies A9 and A31 to HuPBMC and Jurkat cells. FIG. 7A shows the results of the binding activity of the candidate antibodies to HuPBMC cells. FIG. 7B shows the results of the binding activity of the candidate antibodies to Jurkat cells.

FIGS. 8A and 8B show the results of the blocking effects of the candidate antibodies A9 and A31 on the binding of HuCD100 to HuPlexin receptors. FIG. 8A shows the results of the blocking effects of the candidate antibodies on the binding of HuCD100 to HuPlexin-B1-HEK293 cells. FIG. 8B shows the results of the blocking effects of the candidate antibodies on the binding of HuCD100 to HuPlexin-B1-HEK293 cells.

FIGS. 9A-9C show the ELISA detection results of the binding activity of the candidate humanized antibodies A9-VHH1, A9-VHH2, A9-VHH3 and A31-VHH1 to antigen proteins from different species. FIG. 9A shows the results of the binding activity of the candidate humanized antibodies to HuCD100-His antigen protein. FIG. 9B shows the results of the binding activity of the candidate humanized antibodies to MusCD100-His antigen protein. FIG. 9C shows the results of the binding activity of the candidate humanized antibodies to

CynoCD100-His antigen protein.

FIGS. 10A-10F show the FACS detection results of the binding activity of the candidate antibodies to overexpression cell lines from different species. FIG. 10A shows the results of the binding activity of humanized antibodies A9-VHH1, A9-VHH2 and A9-VHH3 and antibody A9 to HuCD100-HEK293 overexpression cell lines. FIG. 10B shows the results of the binding activity of humanized antibodies A9-VHH1, A9-VHH2 and A9-VHH3 and antibody A9 to MusCD100-HEK293 overexpression cell lines. FIG. 10C shows the results of the binding activity of humanized antibodies A9-VHH1, A9-VHH2 and A9-VHH3 and antibody A9 to CynoCD100-HEK293 overexpression cell lines. FIG. 10D shows the results of the binding activity of humanized antibody A31-VHH1 and antibody A31 to HuCD100-HEK293 overexpression cell line. FIG. 10E shows the results of the binding activity of humanized antibody A31-VHH1 and antibody A31 to MusCD100-HEK293 overexpression cell line. FIG. 10F shows the results of the binding activity of humanized antibody A31-VHH1 and antibody A31 to CynoCD100-HEK293 overexpression cell line.

FIGS. 11A-11D show the FACS detection results of the binding activity of the candidate antibodies to HuPBMC and Jurkat cells. FIG. 11A shows the results of the binding activity of humanized antibodies A9-VHH1, A9-VHH2 and A9-VHH3 and antibody A9 to HuPBMC cells. FIG. 11B shows the results of the binding activity of humanized antibody A31-VHH1 and antibody A31 to HuPBMC cells. FIG. 11C shows the results of the binding activity of humanized antibodies A9-VHH1, A9-VHH2 and A9-VHH3 and antibody A9 to Jurkat cells. FIG. 11D shows the results of the binding activity of humanized antibody A31-VHH1 and antibody A31 to Jurkat cells.

FIGS. 12A-12F show the results of the blocking effects of the candidate antibodies on the binding of CD100 to Plexin receptors from different species. FIG. 12A shows the results of the blocking effects of the humanized antibodies A9-VHH1, A9-VHH2 and A9-VHH3 and antibody A9 on the binding of HuCD100 to HuPlexin-B1-HEK293 cells. FIG. 12B shows the results of the blocking effects of the humanized antibody A31-VHH1 and antibody A31 on the binding of HuCD100 to HuPlexin-B1-HEK293 cells. FIG. 12C shows the results of the blocking effects of the humanized antibodies A9-VHH1, A9-VHH2 and A9-VHH3 and antibody A9 on the binding of HuCD100 to HuPlexin-B2-HEK293 cells. FIG. 12D shows the results of the blocking effects of the humanized antibody A31-VHH1 and antibody A31 on the binding of HuCD100 to HuPlexin-B2-HEK293 cells. FIG. 12E shows the results of the blocking effects of the humanized antibodies A9-VHH1, A9-VHH2 and A9-VHH3 and antibody A9 on the binding of MusCD100 to MusPlexin-B1-HEK293 cells. FIG. 12F shows the results of the blocking effects of the humanized antibody A31-VHH1 and antibody A31 on the binding of MusCD100 to MusPlexin-B1-HEK293 cells.

FIG. 13 shows the detection results of inhibition of MDSC proliferation for the candidate antibodies.

FIGS. 14A and 14B show the FACS detection results of the binding activity of anti-PD-L1 antibody m18 to overexpression cell line (human PD-L1-CHO cells) and PD-L1 positive cells (HCC827 cells). FIG. 14A shows the results of the binding activity of antibody m18 to human PD-L1-CHO cells. FIG. 14B shows the results of the binding activity of antibody m18 to HCC827 cells.

FIGS. 15A and 15B show the FACS detection results of the binding activity of anti-PD-L1 antibody m18 to overexpression cell lines from different species. FIG. 15A shows the results of the binding activity of antibody m18 to mouse PD-L1-CHO cells. FIG. 15B shows the results of the binding activity of antibody m18 to cynomolgus monkey PD-L1-CHO cells.

FIG. 16 shows the ELISA detection results of the specific binding activity of anti-PD-L1 antibody m18 to B7-H1 and proteins within the same family.

FIGS. 17A and 17B show the detection results of the tumor inhibition effects. FIG. 17A shows the results of the changes in tumor volume. FIG. 17B shows the results of the change in body weight.

### DETAILED DESCRIPTION

### Definitions

In the present invention, all scientific and technical terms used herein have the meanings typically understood by those skilled in the art unless otherwise specified. In addition, the terms and laboratory operation steps related to the protein and nucleic acid chemistry, molecular biology, cell and tissue culture, microbiology and immunology used herein are terms and conventional steps that are widely used in the corresponding art. To better understand the present invention, definitions and explanations of related terms are provided below.

As used herein, “at least one” or “one or more” may denote 1, 2, 3, 4, 5, 6, 7, 8 or more.

As used herein, the expressions “including”, “comprising”, “containing” and “having” are open-ended and indicate that the listed elements, steps or components are included, but additional unlisted elements, steps or components are not excluded. The expression “consisting of” does not include any unspecified element, step or component. The expression “consisting essentially of” means that the range is limited to the specified elements, steps or components, and optionally, elements, steps or components that do not significantly affect the basic and novel features of the claimed subject matter. It should be understood that the expressions “consisting essentially of” and “consisting of” are encompassed in the meaning of the expression “including”.

As used herein, the conjunctive term “and/or” between multiple recited elements should be understood to include both individual and combined options. In other words, “and/or” includes “and” as well as “or”. For example, A and/or B includes A, B, and A+B, A, B, and/or C includes A, B, C, and any combination thereof, such as A+B, A+C, B+C, and A+B+C. More elements defined by “and/or” are understood in a similar manner, including any of them and any combination thereof.

Unless otherwise specified, any numerical value or numerical range, such as a concentration or concentration range, shall be understood as being modified by the term “about” in any case. Thus, numerical values generally include +10% of the stated value. For example, a concentration of 1 mg/mL includes 0.9 mg/mL to 1.1 mg/mL. Similarly, a concentration range of 1% to 10% (w/v) includes 0.9% (w/v) to 11% (w/v). As used herein, the use of a numerical range explicitly includes all possible subranges, all individual values within the range, including integers and fractions within the range, unless the context explicitly indicates otherwise.

As used herein, an “antibody” refers to an immunoglobulin or a fragment thereof, which specifically binds to an antigen epitope through at least one antigen binding site. Antibodies encompass antibody fragments. As used herein, the term “antibody” includes synthetic antibodies, recombinantly produced antibodies, multispecific antibodies (e.g., bispecific antibodies), human antibodies, non-human antibodies, humanized antibodies, nanobodies, chimeric antibodies, intracellular antibodies, and antibody fragments, such as but not limited to Fab fragments, Fab′ fragments, F(ab′)₂ fragments, Fv fragments, disulfide-linked Fv (dsFv), Fd fragments, Fd′ fragments, single-chain Fv (scFv), single-chain Fab (scFab), diabodies, anti-idiotypic (anti-Id) antibodies, or antigen-binding fragments of any of the aforementioned antibodies. Antibodies provided herein include members of any immunoglobulin type (e.g., IgG, IgM, IgD, IgE, IgA, and IgY), any class (e.g., IgG1, IgG2, IgG3, IgG4, IgA1, and IgA2), or subclass (e.g., IgG2a and IgG2b). In a preferred embodiment, the antibody of the present invention is a nanobody.

As used herein, an “antibody fragment” or “antigen-binding fragment” of an antibody refers to any portion of a full-length antibody that is less than full-length but contains at least the variable region portion of the antibody that binds to the antigen (e.g., one or more CDRs and/or one or more antibody-binding sites) and thus retains binding specificity and at least partial specific binding ability of the full-length antibody. Accordingly, an antigen-binding fragment refers to an antibody fragment containing an antigen-binding portion that binds the same antigen as the antibody from which the fragment is derived. Antibody fragments include derivatives of antibodies produced by enzymatic treatment of full-length antibodies, as well as synthetically produced derivatives, such as recombinantly produced derivatives. Antibodies include antibody fragments. Examples of antibody fragments include, but are not limited to, Fab, Fab′, F(ab′)_{2,} single-chain Fv (scFv), Fv, dsFv, diabodies, Fd, and Fd′ fragments, and other fragments, including modified fragments (see, e.g., Methods in Molecular Biology, Vol 207: Recombinant Antibodies for Cancer Therapy Methods and Protocols (2003); Chapter 1; p 3-25, Kipriyanov). The fragments may include multiple chains linked together, such as via disulfide bonds and/or peptide linkers. Antibody fragments generally contain at least or about 50 amino acids, and typically at least or about 200 amino acids. Antigen-binding fragments include any antibody fragment that, when inserted into an antibody framework (e.g., by replacing corresponding regions), yields an antibody that immunospecifically binds to an antigen.

As used herein, an “immunoglobulin single variable domain” or “single variable domain” refers to a single variable region (variable domain) having antigen-binding activity. Unlike conventional antibodies, where a functional antigen-binding unit is composed of a pair of VH and VL, a single variable domain can form a functional antigen-binding unit alone. Single variable domains can be derived from naturally occurring light chain-free antibodies, such as the variable domain of heavy chain of heavy-chain antibody (VHH) from camelids (e.g., camels and alpacas) and the single variable domain of the new antigen receptor (IgNAR variable single-domain, VNAR) from sharks, or can be screened from full-length antibodies, such as the light-chain variable domain and heavy-chain variable domain having antigen-binding activity in human antibodies. A VHH typically contains three highly variable “complementarity-determining regions (CDRs)” and four relatively conserved “framework regions (FRs)”, and they are linked in the order of FR1-CDR1-FR2-CDR2-FR3-CDR3-FR4 from the N-terminus to the C-terminus.

As used herein, a “single domain antibody (sdAb)” or a “nanobody” refers to an antibody comprising a single immunoglobulin variable domain (single variable domain) as a functional antigen binding fragment. Similar to the variable region of a full-length antibody, a single variable domain typically comprises CDR1, CDR2 and CDR3 that form an antigen binding site and a supporting framework region. Unlike full-length antibodies, which typically contain two heavy chains and two light chains, single domain antibodies generally contain a single peptide chain composed of a single variable domain, with a molecular weight of only approximately 15 kDa. The single variable domain can be, for example, the variable domain of an alpaca heavy-chain antibody (variable domain of heavy-chain antibody, VHH), a shark IgNAR variable domain, or a human light-chain antibody variable domain.

As used herein, the terms “heavy-chain-only antibody” and “heavy-chain antibody” are used interchangeably and exist in their broadest sense, referring to antibodies lacking conventional antibody light chains, which contain only a VHH and a heavy-chain constant region without CH1 (e.g., Fc fragment).

An “Fc fragment” generally refers to the crystallizable fragment obtained by papain digestion of conventional antibodies or heavy-chain antibodies. In general, the Fc fragment of IgG and heavy-chain antibodies may contain part of the hinge region, CH2, and CH3. Herein, the Fc fragment may contain at least part of the hinge region (e.g., all or part of the hinge region), CH2, and CH3.

Unless otherwise indicated, the amino acid sequences of CDRs herein are shown in accordance with the AbM definition rules (the sequences in the claims of the present invention are also shown in accordance with the AbM definition rules). However, it is well known to those skilled in the art that multiple methods can be used in the art to define the CDRs of an antibody, such as Chothia based on the three-dimensional structure of the antibody and the topology of the CDR loops (see, e.g., Chothia, C. et al., Nature, 342, 877-883 (1989); and Al-Lazikani, B. et al.., J. Mol. Biol., 273, 927-948 (1997)), Kabat based on the variability of antibody sequences (see, e.g., Kabat, E. A. et al. (1991) Sequences of Proteins of Immunological Interest, Fifth Edition, U.S. Department of Health and Human Services, NIH Publication No. 91-3242), AbM (Martin, A. C. R. and J. Allen (2007) “Bioinformatics tools for antibody engineering,” in S. Dübel (ed.), Handbook of Therapeutic Antibodies. Weinheim: Wiley-VCH Verlag, pp. 95-118), Contact (MacCallum, R. M. et al., (1996) J. Mol. Biol. 262:732-745), IMGT (Lefranc, M.-P., 2011 (6), IMGT, the International ImMunoGeneTics Information System Cold Spring Harb Protoc.; and Lefranc, M.-P. et al., Dev. Comp. Immunol., 27, 55-77 (2003)), and North CDR definition based on affinity propagation clustering using a large number of crystal structures. Herein, multiple CDR definition systems may be used for the same variable region, such as Chothia, Abm, Kabat, Contact, and IMGT. Those skilled in the art should understand that although CDRs defined by different definition systems may differ, CDRs corresponding to the same definition system represent effective antigen-binding sites capable of binding to epitopes. Unless otherwise specified, the terms “CDR” and “complementarity-determining region” of a given antibody or its region (e.g., variable region) should be understood to encompass complementarity-determining regions defined by any of the known schemes described above in the present invention. Although the scope of protection as claimed in the claims of the present invention is based on the sequences shown according to the Abm definition rules, the corresponding amino acid sequences according to other CDR definition rules should also fall within the protection scope of the present invention.

Therefore, when it comes to defining an antibody with specific CDR sequences defined in the present invention, the scope of the antibody also encompasses such an antibody whose variable region sequences comprise the specific CDR sequences but whose claimed CDR boundaries are different from the specific CDR boundaries defined by the present invention due to the application of a different scheme (for example, rules of different assignment systems or their combinations).

As used herein, the terms “framework region” and “scaffold region” may be used interchangeably. As used herein, the term “framework region” residue, “scaffold region” residue or “FR” residue refers to those amino acid residues in the variable region of the antibody other than the CDR sequences defined above.

The term “disulfide bond” as used herein includes a covalent bond formed between two sulfur atoms. The amino acid cysteine contains a sulfhydryl group capable of forming a disulfide bond or bridging with a second sulfhydryl group.

The term “chimeric antibody” as used herein refers to an antibody in which the immunoreactive region or site is obtained from or derived from a first species, while the constant region (which may be intact, partial, or modified) is obtained from a second species. In some embodiments, the target-binding region or site will be of non-human origin (e.g., murine or primate) and the constant region will be human.

As used herein, the term “humanized antibody” refers to a non-human antibody modified to increase sequence homology with human antibodies. Humanized antibodies generally retain the antigen-binding ability of the non-human antibody from which they are derived and have lower immunogenicity in humans. Humanized antibodies can be obtained by antibody engineering of any non-human species antibody or antibodies containing sequences from non-human species (e.g., chimeric antibodies). Non-human species may include, for example, mice, rats, rabbits, alpacas, sharks, or non-human primates. Techniques for obtaining humanized antibodies from non-human antibodies are well known to those skilled in the art. For example, CDR sequences of non-human antibodies (e.g., murine antibodies) are grafted into human antibody framework regions. In some cases, to maintain the antigen-binding ability and/or stability of humanized antibodies, key amino acid residues of the framework sequences of non-human antibodies (e.g., murine antibodies) may be retained in the human antibody framework regions, i.e., “back mutations” (see, e.g., Morrison et al. (1984) Proc. Natl. Acad. Sci. 81(21): 6851-6855; Neuberger et al. (1984) Nature 312:604-608).

As used herein, the terms “percentage (%) sequence identity” and “sequence identity” of amino acid sequences have a well-recognized meaning in the art and refer to the percentage of the identical residues of two polypeptide sequences determined by sequence alignment (for example, by manual inspection or a well-known algorithm). This can be determined using methods known to those skilled in the art, for example, using a publicly available computer software such as BLAST, BLAST-2, Clustal Omega and FASTA software.

An amino acid sequence “originated from” or “derived from” a reference amino acid sequence herein is partially or completely identical or homologous to the reference amino acid sequence. For example, an amino acid sequence derived from a human immunoglobulin heavy chain constant region may have at least 80%, at least 85%, at least 90%, at least 91%, at least 92%, at least 93%, at least 94%, at least 95%, at least 96%, at least 97%, at least 98%, at least 99% or 100% sequence identity to the wild-type sequence of the human immunoglobulin heavy chain constant region from which the amino acid sequence is derived.

Non-critical regions in a polypeptide (for example, non-critical amino acids of CDR regions and framework regions of an antibody, amino acids of constant regions) can be modified, for example, by substitution, addition and/or deletion of one or more amino acids without altering the function of the polypeptide. Suitable conservative amino acid substitutions in peptides or proteins are known to those skilled in the art and can generally be made without altering the biological activity of the resulting molecule. Typically, those skilled in the art recognize that single amino acid substitutions in non-essential regions of a polypeptide generally do not alter biological activity (see, e.g., Watson et al., Molecular Biology of the Gene, 4th Edition, 1987, The Benjamin/Cummings Pub. Co., p. 224).

“Affinity” or “binding affinity” is used to measure the strength of non-covalent interaction between an antibody and an antigen. Affinity can be determined by conventional techniques known in the art, such as biolayer interferometry (e.g., using an Octet Fortebio detection system), radioimmunoassay, surface plasmon resonance, enzyme-linked immunosorbent assay (ELISA), or flow cytometry (FACS).

“Specific binding” generally denotes that a binding molecule, such as an antibody or a fragment, variant, or derivative thereof, binds an epitope via its antigen-binding domain, and such binding requires some complementarity between the antigen-binding domain and the epitope. By this definition, a binding molecule is said to “specifically bind” to an epitope when it binds to the epitope via its antigen-binding domain more readily than it binds to random, unrelated epitopes. The term “specificity” is used herein to qualitatively analyze the relative affinity of a particular antibody binding to a particular epitope. For example, binding molecule “A” may be considered to have higher specificity for a given epitope than binding molecule “B”, or it may be said that binding molecule “A” binds to epitope “C” with higher specificity than its specificity for related epitope “D”.

If a binding molecule, such as an antibody or a fragment, variant, or derivative thereof, preferentially binds to an epitope to an extent that it blocks the binding of a reference antibody or an antigen-binding fragment to that epitope, the binding molecule (e.g., the antibody or fragment, variant, or derivative thereof) can be said to competitively inhibit the binding of the reference antibody or antigen-binding fragment to the given epitope. Competitive inhibition can be determined by any method known in the art, such as competitive ELISA assays. A binding molecule can be said to competitively inhibit at least 90%, at least 80%, at least 70%, at least 60%, or at least 50% of the binding of a reference antibody or an antigen-binding fragment to a given epitope.

As used herein, the term “PD-L1” refers to programmed cell death ligand 1 (PD-L1, see, e.g., Freeman et al. Engagement of the PD-1 immunoinhibitory receptor by a novel B7 family member leads to negative regulation of lymphocyte activation. J Exp Med. 2000 Oct. 2; 192(7)). PD-L1 belongs to the B7 family. Alternative names or synonyms for PD-L1 include PDCD1L1, PDL1, B7 homolog 1 (B7-H1), cluster of differentiation 274 (CD274), or B7-H, etc. The representative amino acid sequence of human PD-L1 is disclosed under NCBI accession number NP_054862.1, and the representative nucleic acid sequence encoding human PD-L1 is shown under NCBI accession number NM_014143.4. PD-L1 is expressed in placenta, spleen, lymph nodes, thymus, heart, fetal liver, and is also found in many tumor or cancer cells. PD-L1 binds to its receptors PD-1 or B7-1, which are expressed on activated T cells, B cells, and myeloid cells. Binding of PD-L1 to its receptors induces signal transduction to inhibit TCR-mediated cytokine production and T cell proliferation activation. Thus, PD-L1 plays a major role in suppressing the immune system during specific events (e.g., pregnancy, autoimmune diseases, and tissue allografts) and is believed to allow tumor or cancer cells to bypass immune checkpoints and evade immune responses.

As used herein, the term “B7 family” refers to a class of structurally similar co-stimulatory factors involved in the body's immune processes. It belongs to the immunoglobulin class and is associated with the activation of T and B cells and body's immunity.

As used herein, the terms “polynucleotide” and “nucleic acid” are used interchangeably to refer to polymers of deoxyribonucleotides (deoxyribonucleic acid, DNA) or polymers of ribonucleotides (ribonucleic acid, RNA). “Polynucleotide sequence”, “nucleic acid sequence”, and “nucleotide sequence” are used interchangeably to denote the ordering of nucleotides in a polynucleotide. Those skilled in the art will understand that the DNA coding strand (sense strand) and the RNA it encodes can be regarded as having the same nucleotide sequence, with deoxythymidine in the DNA coding strand sequence corresponding to uridine in the encoded RNA sequence.

As used herein, an isolated nucleic acid molecule is a nucleic acid molecule separated from other nucleic acid molecules present in its natural source. An “isolated” nucleic acid molecule such as a cDNA molecule may be substantially free of other cellular material or culture medium when produced by recombinant techniques, or substantially free of chemical precursors or other chemical components when chemically synthesized. Exemplary isolated nucleic acid molecules provided herein include isolated nucleic acid molecules encoding the provided antibodies or antigen-binding fragments.

As used herein, the term “expression” includes transcription and/or translation of a nucleotide sequence. Thus, expression may involve production of transcripts and/or polypeptides.

As used herein, a “vector” is a vehicle for introducing an exogenous polynucleotide into a host cell, enabling the amplification and expression of the exogenous polynucleotide when the vector is transformed into a suitable host cell. Vectors are typically maintained in an episomal state, but may be designed to allow the integration of the gene or a part thereof into the chromosome of the genome. As used herein, the definition of a “vector” encompasses a plasmid, a linearized plasmid, a viral vector, a cosmid, a phage vector, a phagemid, an artificial chromosome (for example, a yeast artificial chromosome and a mammalian artificial chromosome), etc. The viral vector includes but is not limited to a retrovirus vector (including a lentivirus vector), an adenovirus vector, an adeno-associated virus vector, a herpes virus vector, a poxvirus vector, a baculovirus vector, etc.

As used herein, a “host cell” refers to a cell used to receive, maintain, replicate, or amplify a vector. Host cells can also be used to express polypeptides encoded by the vector. Upon host cell division, the nucleic acid contained in the vector replicates, thereby amplifying the nucleic acid. Host cells may be eukaryotic or prokaryotic cells. Suitable host cells include but are not limited to CHO cells, various COS cells, HeLa cells, HEK cells such as HEK 293 cells.

The terms such as “treat” or “treatment” or “treating” or “alleviate” or “alleviating” refer to therapeutic measures that cure, slow, reduce symptoms of an existing diagnosed pathological condition or disorder, and/or halt or slow the progression of an existing diagnosed pathological condition or disorder. The terms such as “preventing”, “protecting”, “avoiding”, “arresting” etc., refer to prophylactic or preventive measures to prevent the development of an undiagnosed target pathological condition or disorder. Accordingly, a “subject in need” may include a subject who has already been diagnosed with a disease, a subject who is susceptible to the disease, and a subject who requires prevention against the disease.

As used herein, “efficacy” refers to an effect resulting from treatment of an individual that changes, typically improves or ameliorates, symptoms of a disease or condition, or cures the disease or condition.

The term “therapeutically effective amount” refers to an amount of an antibody, polypeptide, polynucleotide, small organic molecule, or other drug effective for “treating” a disease or disorder in a subject or mammal. In the context of cancer, a therapeutically effective amount of a medicament may reduce the number of cancer cells; arrest or stop cancer cell division; reduce or halt tumor size increase; inhibit, e.g., suppress, block, prevent, stop, delay, or reverse cancer cell infiltration into surrounding organs, including, e.g., cancer metastasis to soft tissues and bone; inhibit, e.g., suppress, block, prevent, shrink, stop, delay, or reverse tumor metastasis; inhibit, e.g., suppress, block, prevent, stop, delay, or reverse tumor growth; alleviate to some extent one or more symptoms associated with cancer, reduce morbidity and mortality; improve quality of life; or a combination of these effects. To the extent the medicament prevents the growth and/or kills existing cancer cells, it may be referred to as cytostatic and/or cytotoxic.

As used herein, the term “subject” refers to a mammal, such as a human.

As used herein, antibody designations (such as A9-VHH, A31-VHH, A9, A31, A9-VHH1, A9-VHH2, A9-VHH3, A31-VHH1, m18-VHH or m18) are merely used to distinguish or identify antibodies or products and are not intended to indicate that such identifiers are characteristics of the antibodies or products of the invention. Those skilled in the art shall understand that, for example, for the purpose of distinguishing or identification, other antibodies or products may also use such identifiers, but do not refer to the same or equivalent antibodies or products. Likewise, similar numbers or identifiers used in the Examples are only for convenience of illustration, and the antibodies or products of the present invention are defined by the features described in the appended claims.

### Anti-CD100 Antibodies or Antigen-Binding Fragments Thereof

The present invention provides an antibody (CD100 antibody) or an antigen-binding fragment thereof against CD100, wherein the antibody or antigen-binding fragment thereof specifically recognizes and binds to CD100.

In some embodiments, the anti-CD100 antibody or antigen-binding fragment thereof of the present invention is a single domain antibody, a heavy chain antibody, a humanized antibody or a chimeric antibody.

In an embodiment, the antibody or antigen-binding fragment thereof has at least one of the following characteristics:
1) having affinity for CD100 protein;
2) having affinity for CD100-positive cells;
3) blocking the binding of CD100 to Plexin-B1 or Plexin-B2;
4) inhibiting proliferation of MDSC cells;
5) inhibiting tumor growth.

In some embodiments, the antibody or antigen-binding fragment thereof of the present invention is capable of specifically binding to CD100 (e.g., human CD100) and blocking its interaction with Plexin-B1 or Plexin-B2.

In some embodiments, the targeted tumors include, but are not limited to, those described below with respect to neoplastic diseases. In other embodiments, the antibody or antigen-binding fragment thereof of the present invention is capable of inhibiting tumor growth by at least about 10%, preferably at least about 20%, more preferably at least about 30%, more preferably at least about 40%, more preferably at least about 50%, more preferably at least about 60%, more preferably at least about 70%, more preferably at least about 80%.

In an embodiment, the antibody or antigen-binding fragment thereof against CD100 comprises an immunoglobulin single variable domain, wherein the single variable domain comprises three CDR sequences in the amino acid sequence of the single variable domain as set forth in SEQ ID NO: 4 or a variant thereof; wherein the variant differs from the CDR sequence from which it is derived by addition, deletion or substitution of no more than 2 amino acids.

In an embodiment, the antibody or antigen-binding fragment thereof against CD100 comprises an immunoglobulin single variable domain, wherein the single variable domain comprises three CDR sequences in the amino acid sequence of the single variable domain as set forth in SEQ ID NO: 8 or a variant thereof; wherein the variant differs from the CDR sequence from which it is derived by addition, deletion or substitution of no more than 2 amino acids.

In an embodiment, the single variable domain comprises: (a) a CDR1 sequence as set forth in SEQ ID NO: 1; a CDR2 sequence as set forth in SEQ ID NO: 2; and a CDR3 sequence as set forth in SEQ ID NO: 3; or (b) a CDR1 sequence as set forth in SEQ ID NO: 5; a CDR2 sequence as set forth in SEQ ID NO: 6; and a CDR3 sequence as set forth in SEQ ID NO: 7.

In an embodiment, the immunoglobulin single variable domain is a variable domain of a heavy chain antibody (VHH).

In an embodiment, the antibody or antigen-binding fragment thereof comprises an immunoglobulin single variable domain, wherein the single variable domain comprises an amino acid sequence as set forth in SEQ ID NO: 4 or an amino acid sequence having at least 85%, at least 90%, at least 95% or higher sequence identity to SEQ ID NO: 4.

In an embodiment, the antibody or antigen-binding fragment thereof comprises an immunoglobulin single variable domain, wherein the single variable domain comprises an amino acid sequence as set forth in SEQ ID NO: 8 or an amino acid sequence having at least 85%, at least 90%, at least 95% or higher sequence identity to SEQ ID NO: 8.

In an embodiment, the anti-CD100 antibody comprises anti-CD100 antibody A9-VHH or A31-VHH.

### Antibody A9-VHH

In an aspect, the present invention provides the antibody A9-VHH or antigen-binding fragment thereof against CD100,
wherein the antibody comprises an immunoglobulin single variable domain, wherein
the single variable domain comprises:
a CDR1 sequence as set forth in SEQ ID NO: 1,
a CDR2 sequence as set forth in SEQ ID NO: 2, and
a CDR3 sequence as set forth in SEQ ID NO: 3.

In an embodiment, the anti-CD100 antibody A9-VHH or antigen-binding fragment thereof comprises an immunoglobulin single variable domain,
wherein the single variable domain comprises an amino acid sequence as set forth in SEQ ID NO: 4 or an amino acid sequence having at least 85%, at least 90%, at least 95% or higher sequence identity to SEQ ID NO: 4.

### Antibody A31-VHH

In another aspect, the present invention provides the antibody A31-VHH or antigen-binding fragment thereof against CD100,
wherein the antibody comprises an immunoglobulin single variable domain, wherein
the single variable domain comprises:
a CDR1 sequence as set forth in SEQ ID NO: 5,
a CDR2 sequence as set forth in SEQ ID NO: 6, and
a CDR3 sequence as set forth in SEQ ID NO: 7.

In an embodiment, the anti-CD100 antibody A31-VHH or antigen-binding fragment thereof comprises an immunoglobulin single variable domain,
wherein the single variable domain comprises an amino acid sequence as set forth in SEQ ID NO: 8 or an amino acid sequence having at least 85%, at least 90%, at least 95% or higher sequence identity to SEQ ID NO: 8.

In an embodiment, the immunoglobin single variable domain of the CD100 antibody or antigen-binding fragment thereof is capable of being fused to an additional molecule.

In a preferred embodiment, the additional molecule is an Fc domain of an immunoglobulin.

In a preferred embodiment, the additional molecule is an Fc domain of an immunoglobulin G4 (IgG4SP).

In an embodiment, the additional molecule comprises the amino acid sequence as set forth in SEQ ID NO: 19.

In an embodiment, the anti-CD100 antibody comprises an antibody A9 or an antibody A31.

### Antibody A9

In some specific embodiments, the antibody A9 or antigen-binding fragment thereof comprises an immunoglobulin single variable domain, wherein the single variable domain comprises the CDR1, CDR2, and CDR3 sequences contained in the antibody A9-VHH or antigen-binding fragment thereof as defined herein.

In an embodiment, the anti-CD100 antibody A9 or antigen-binding fragment thereof comprises an immunoglobulin single variable domain,
wherein the single variable domain comprises an amino acid sequence as set forth in SEQ ID NO: 4 or an amino acid sequence having at least 85%, at least 90%, at least 95% or higher sequence identity to SEQ ID NO: 4.

In an embodiment, the anti-CD100 antibody A9 or antigen-binding fragment thereof further comprises an Fc fragment of a human IgG4SP.

In an embodiment, the antibody A9 or antigen-binding fragment thereof further comprises the amino acid sequence as set forth in SEQ ID NO: 19.

In an embodiment, the antibody A9 or antigen-binding fragment thereof comprises an amino acid sequence as set forth in SEQ ID NO: 9 or an amino acid sequence having at least 85%, at least 90%, at least 95% or higher sequence identity to SEQ ID NO: 9.

### Antibody A31

In some specific embodiments, the antibody A31 or antigen-binding fragment thereof comprises an immunoglobulin single variable domain, wherein the single variable domain comprises the CDR1, CDR2 and CDR3 sequences contained in the antibody A31-VHH or antigen-binding fragment thereof as defined herein.

In an embodiment, the anti-CD100 antibody A31 or antigen-binding fragment thereof comprises an immunoglobulin single variable domain,
wherein the single variable domain comprises an amino acid sequence as set forth in SEQ ID NO: 8 or an amino acid sequence having at least 85%, at least 90%, at least 95% or higher sequence identity to SEQ ID NO: 8.

In an embodiment, the anti-CD100 antibody A31 or antigen-binding fragment thereof further comprises an Fc fragment of a human IgG4SP.

In an embodiment, the antibody A31 or antigen-binding fragment thereof further comprises the amino acid sequence as set forth in SEQ ID NO: 19.

In an embodiment, the antibody A31 or antigen-binding fragment thereof comprises an amino acid sequence as set forth in SEQ ID NO: 10 or an amino acid sequence having at least 85%, at least 90%, at least 95% or higher sequence identity to SEQ ID NO: 10.

In an embodiment, the anti-CD100 antibody or antigen-binding fragment thereof is a humanized antibody or an antigen-binding fragment thereof. In such embodiments, the heavy chain framework region and/or light chain framework region of the antibody or antigen-binding fragment thereof of the present invention may comprise one or more non-human (e.g., murine) amino acid residues, e.g., the heavy chain framework region and/or light chain framework region may comprise one or more amino acid back mutations, wherein the corresponding murine amino acid residues are present in these back mutations.

In an embodiment, the anti-CD100 antibody comprises antibody A9-VHH1, A9-VHH2, A9-VHH3 or A31-VHH1.

### Antibody A9-VHH1

In some specific embodiments, the antibody A9-VHH1 or antigen-binding fragment thereof comprises an immunoglobulin single variable domain, wherein the single variable domain comprises the CDR1, CDR2 and CDR3 sequences contained in the antibody A9-VHH or antigen-binding fragment thereof as defined herein.

In an embodiment, the anti-CD100 antibody A9-VHH1 or antigen-binding fragment thereof comprises an immunoglobulin single variable domain,
wherein the single variable domain comprises an amino acid sequence as set forth in SEQ ID NO: 11 or an amino acid sequence having at least 85%, at least 90%, at least 95% or higher sequence identity to SEQ ID NO: 11.

In an embodiment, the anti-CD100 antibody A9-VHH1 or antigen-binding fragment thereof further comprises an Fc fragment of a human IgG4SP.

In an embodiment, the anti-CD100 antibody A9-VHH1 or antigen-binding fragment thereof comprises an amino acid sequence as set forth in SEQ ID NO: 12 or an amino acid sequence having at least 85%, at least 90%, at least 95% or higher sequence identity to SEQ ID NO: 12.

### Antibody A9-VHH2

In some specific embodiments, the antibody A9-VHH2 or antigen-binding fragment thereof comprises an immunoglobulin single variable domain, wherein the single variable domain comprises the CDR1, CDR2 and CDR3 sequences contained in the antibody A9-VHH or antigen-binding fragment thereof as defined herein.

In an embodiment, the anti-CD100 antibody A9-VHH2 or antigen-binding fragment thereof comprises an immunoglobulin single variable domain,
wherein the single variable domain comprises an amino acid sequence as set forth in SEQ ID NO: 13 or an amino acid sequence having at least 85%, at least 90%, at least 95% or higher sequence identity to SEQ ID NO: 13.

In an embodiment, the anti-CD100 antibody A9-VHH2 or antigen-binding fragment thereof further comprises an Fc fragment of a human IgG4SP.

In an embodiment, the anti-CD100 antibody A9-VHH2 or antigen-binding fragment thereof comprises an amino acid sequence as set forth in SEQ ID NO: 14 or an amino acid sequence having at least 85%, at least 90%, at least 95% or higher sequence identity to SEQ ID NO: 14.

### Antibody A9-VHH3

In some specific embodiments, the antibody A9-VHH3 or antigen-binding fragment thereof comprises an immunoglobulin single variable domain, wherein the single variable domain comprises the CDR1, CDR2 and CDR3 sequences contained in the antibody A9-VHH or antigen-binding fragment thereof as defined herein.

In an embodiment, the anti-CD100 antibody A9-VHH3 or antigen-binding fragment thereof comprises an immunoglobulin single variable domain,
wherein the single variable domain comprises an amino acid sequence as set forth in SEQ ID NO: 15 or an amino acid sequence having at least 85%, at least 90%, at least 95% or higher sequence identity to SEQ ID NO: 15.

In an embodiment, the anti-CD100 antibody A9-VHH3 or antigen-binding fragment thereof further comprises an Fc fragment of a human IgG4SP.

In an embodiment, the anti-CD100 antibody A9-VHH3 or antigen-binding fragment thereof comprises an amino acid sequence as set forth in SEQ ID NO: 16 or an amino acid sequence having at least 85%, at least 90%, at least 95% or higher sequence identity to SEQ ID NO: 16.

### Antibody A31-VHH1

In some specific embodiments, the antibody A31-VHH1 or antigen-binding fragment thereof comprises an immunoglobulin single variable domain, wherein the single variable domain comprises the CDR1, CDR2 and CDR3 sequences contained in the antibody A31-VHH or antigen-binding fragment thereof as defined herein.

In an embodiment, the anti-CD100 antibody A31-VHH1 or antigen-binding fragment thereof comprises an immunoglobulin single variable domain,
wherein the single variable domain comprises an amino acid sequence as set forth in SEQ ID NO: 17 or an amino acid sequence having at least 85%, at least 90%, at least 95% or higher sequence identity to SEQ ID NO: 17.

In an embodiment, the anti-CD100 antibody A31-VHH1 or antigen-binding fragment thereof further comprises an Fc fragment of a human IgG4SP.

In an embodiment, the anti-CD100 antibody A31-VHH1 or antigen-binding fragment thereof comprises an amino acid sequence as set forth in SEQ ID NO: 18 or an amino acid sequence having at least 85%, at least 90%, at least 95% or higher sequence identity to SEQ ID NO: 18.

### Nucleic Acid Molecules, Vectors and Host Cells

In another aspect, the present invention provides a nucleic acid molecule comprising a polynucleotide sequence encoding the anti-CD100 antibody or antigen-binding fragment thereof of the present invention. In some embodiments, the nucleic acid molecule of the present invention encodes the anti-CD100 antibody or antigen-binding fragment thereof of the present invention.

The nucleic acid molecules of the present invention may be obtained by using methods known in the art. For example, the nucleic acid molecules of the present invention may be isolated from phage display libraries, yeast display libraries, immunized animals, immortalized cells (e.g., murine B cell hybridoma cells, EBV-mediated immortalized B cells), or chemically synthesized. The nucleic acid molecules of the present invention may be codon-optimized for a host cell for expression.

In yet another aspect, the present invention further provides an expression vector comprising the nucleic acid molecule of the present invention. The expression vector may further comprise additional polynucleotide sequences, for example, regulatory sequences and antibiotic resistance genes. The nucleic acid molecule of the present invention may be present in one or more expression vectors. In an embodiment, the nucleic acid molecule of the present invention is prepared as a recombinant nucleic acid. Recombinant nucleic acids may be prepared by using techniques well known in the art, such as chemical synthesis, DNA recombination techniques (e.g., polymerase chain reaction (PCR) techniques), and the like.

The present invention also provides a host cell comprising the nucleic acid molecule or expression vector of the present invention. The nucleic acid molecule or expression vector of the present invention may be introduced into a suitable host cell by using various methods known in the art. Such methods include, but are not limited to, lipofection, electroporation, viral transduction, calcium phosphate transfection, etc.

In a preferred embodiment, the host cell is used to express the anti-CD100 antibody or antigen-binding fragment thereof of the present invention. Examples of the host cell include, but are not limited to, a prokaryotic cell (e.g., bacteria, such as E. coli) and a eukaryotic cell (e.g., yeast, an insect cell, and a mammalian cell). Mammalian host cells suitable for antibody expression include, but are not limited to, human cervical cancer cells (HeLa cells), human embryonic kidney cells (HEK cells, such as HEK 293 cells), Chinese hamster ovary (CHO) cells, and other mammalian cells suitable for antibody expression.

The present invention further provides a method for producing the anti-CD100 antibody or antigen-binding fragment thereof according to the present invention, comprising the following steps:
a) culturing the host cell of the present invention under suitable conditions to express the anti-CD100 antibody or antigen-binding fragment thereof of the present invention; and
b) isolating the antibody or antigen-binding fragment thereof from the host cell or a culture thereof.

### Pharmaceutical Combination

In another aspect, the present invention provides a pharmaceutical combination, which enables the administration of two or more therapeutic or prophylactic agents to a subject through the application of the pharmaceutical combination. The pharmaceutical combination comprises an antibody or an antigen-binding fragment thereof against CD100 and an antibody or an antigen-binding fragment thereof against PD-L1.

In an embodiment, the antibody against CD100 comprises antibody A9-VHH, A31-VHH, A9, A31, A9-VHH1, A9-VHH2, A9-VHH3, A31-VHH1, or a combination thereof.

In an embodiment, the antibody or antigen-binding fragment thereof against PD-L1 specifically recognizes and binds to PD-L1, wherein the anti-PD-L1 antibody or antigen-binding fragment thereof comprises an immunoglobulin single variable domain.

In a preferred embodiment, the immunoglobulin single variable domain comprises:
a CDR1 sequence as set forth in SEQ ID NO: 20,
a CDR2 sequence as set forth in SEQ ID NO: 21, and
a CDR3 sequence as set forth in SEQ ID NO: 22.

In a preferred embodiment, the immunoglobulin single variable domain comprises: 1) the amino acid sequence as set forth in SEQ ID NO: 23; or 2) an amino acid sequence having at least 85%, at least 90%, at least 95% or higher sequence identity to SEQ ID NO: 23.

In an embodiment, the anti-PD-L1 antibody or antigen-binding fragment thereof further comprises an Fc fragment of a human IgG1.

In a preferred embodiment, the anti-PD-L1 antibody or antigen-binding fragment thereof comprises an amino acid sequence as set forth in SEQ ID NO: 24 or an amino acid sequence having at least 85%, at least 90%, at least 95% or higher sequence identity to SEQ ID NO: 24.

In an embodiment, the anti-PD-L1 antibody comprises the anti-PD-L1 antibody m18-VHH or m18.

### Antibody m18-VHH

In an aspect, the present invention provides the antibody m18-VHH or antigen-binding 35 fragment thereof against PD-L1.
wherein the antibody comprises an immunoglobulin single variable domain, wherein
the single variable domain comprises:
a CDR1 sequence as set forth in SEQ ID NO: 20,
a CDR2 sequence as set forth in SEQ ID NO: 21, and
a CDR3 sequence as set forth in SEQ ID NO: 22.

In an embodiment, the anti-PD-L1 antibody m18-VHH comprises an immunoglobulin single variable domain,
wherein the single variable domain comprises an amino acid sequence as set forth in SEQ ID NO: 23 or an amino acid sequence having at least 85%, at least 90%, at least 95% or higher sequence identity to SEQ ID NO: 23.

### Antibody m18

In yet another aspect, the present invention further provides the antibody m18 or antigen-binding fragment thereof against PD-L1,
wherein the antibody comprises an immunoglobulin single variable domain, wherein
the single variable domain comprises:
a CDR1 sequence as set forth in SEQ ID NO: 20,
a CDR2 sequence as set forth in SEQ ID NO: 21, and
a CDR3 sequence as set forth in SEQ ID NO: 22.

In an embodiment, the anti-PD-L1 antibody m18 comprises an immunoglobulin single variable domain,
wherein the single variable domain comprises an amino acid sequence as set forth in SEQ ID NO: 23 or an amino acid sequence having at least 85%, at least 90%, at least 95% or higher sequence identity to SEQ ID NO: 23.

In an embodiment, the anti-PD-L1 antibody m18 further comprises an Fc fragment of a human IgG1, the antibody m18 comprises an amino acid sequence as set forth in SEQ ID NO: 24 or an amino acid sequence having at least 85%, at least 90%, at least 95% or higher sequence identity to SEQ ID NO: 24.

In some embodiments, the anti-PD-L1 antibody or antigen-binding fragment thereof of the present invention is a single domain antibody, a heavy chain antibody, a humanized antibody or a chimeric antibody. Preferably, the antibody or antigen-binding fragment thereof is a human antibody.

In an embodiment, the antibody or antigen-binding fragment thereof has at least one of the following characteristics:
1) having affinity for PD-L1 positive cells;
2) being capable of specifically binding to the PD-L1 protein;
3) inhibiting tumor growth.

In some embodiments, the targeted tumors include, but are not limited to, those described below with respect to neoplastic diseases. In other embodiments, the antibody or antigen-binding fragment thereof of the present invention is capable of inhibiting tumor growth by at least about 10%, preferably at least about 20%, more preferably at least about 30%, more preferably at least about 40%, more preferably at least about 50%, more preferably at least about 60%, more preferably at least about 70%, more preferably at least about 80%.

In an embodiment, the pharmaceutical combination comprises an antibody against CD100 comprising antibody A9-VHH, A31-VHH, A9, A31, A9-VHH1, A9-VHH2, A9-VHH3, A31-VHH1, or a combination thereof; and the pharmaceutical combination comprises an antibody against PD-L1 comprising the antibody m18-VHH or m18.

In a preferred embodiment, the pharmaceutical combination comprises the anti-CD100 antibody A9-VHH1, A9-VHH2, A9-VHH3, A31-VHH1 or a combination thereof and the anti-PD-L1 antibody m18-VHH or m18.

In a preferred embodiment, the pharmaceutical combination comprises the anti-CD100 antibody A9-VHH2 and the anti-PD-L1 antibody m18-VHH or m18.

In an embodiment, the pharmaceutical combination may be a pharmaceutical composition or a kit.

### Pharmaceutical Composition

The present invention further provides a pharmaceutical composition comprising the anti-CD100 antibody or antigen-binding fragment thereof of the present invention and a pharmaceutically acceptable carrier. The aforementioned anti-CD100 antibody includes anti-CD100 antibody A9-VHH, A31-VHH, A9, A31, A9-VHH1, A9-VHH2, A9-VHH3, A31-VHH1, or a combination thereof, particularly anti-CD100 antibody A9-VHH1, A9-VHH2, A9-VHH3 or A31-VHH1. In an embodiment, the pharmaceutical composition comprises a combination of anti-CD100 antibody A9-VHH, A31-VHH, A9, A31, A9-VHH1, A9-VHH2, A9-VHH3, or A31-VHH1 and anti-PD-L1 antibody m18 or m18-VHH. In a preferred embodiment, the pharmaceutical composition comprises a combination of anti-CD100 antibody A9-VHH1, A9-VHH2, A9-VHH3 or A31-VHH1 and anti-PD-L1 antibody m18 or m18-VHH. In a preferred embodiment, the pharmaceutical composition comprises a combination of anti-CD100 antibody A9-VHH2 and anti-PD-L1 antibody m18 or m18-VHH.

The pharmaceutical compositions provided herein may be formulated in a variety of dosage forms, including but not limited to solid, semi-solid, liquid, powder or lyophilized forms. For compositions comprising the antibody or antigen-binding fragment thereof, the preferred dosage forms may generally be, for example, injectable solutions and lyophilized powders.

The anti-CD100 antibody or antigen-binding fragment thereof, pharmaceutical composition or pharmaceutical combination provided herein may be administered to a subject by any method known in the art, such as by systemic or local administration. Routes of administration include but are not limited to parenteral (e.g., intravenous, intraperitoneal, intradermal, intramuscular, subcutaneous or intracavitary), local (e.g., intratumoral), epidural or mucosal (e.g., intranasal, oral, vaginal, rectal, sublingual or topical) routes. Preferably, the anti-CD100 antibody or antigen-binding fragment thereof, pharmaceutical composition or pharmaceutical combination is suitable for intravenous, intramuscular, subcutaneous, parenteral, spinal or epidermal administration (such as via injection or infusion). The administration method may be, for example, injection or infusion.

Those skilled in the art will appreciate that the exact dosage will depend on various factors, such as the pharmacokinetic properties of the pharmaceutical composition, the duration of the treatment, the excretion rate of the specific compound, the purpose of the treatment, the route of administration, and the condition of the subject (e.g., the age, health status, body weight, gender, diet, medical history of the patient), and other factors well-known in the medical field. As a general guide, the dosage range for the anti-CD100 antibody or antigen-binding fragment thereof, the pharmaceutical composition or the pharmaceutical combination of the present invention is about 0.0001-100 mg/kg, more typically 0.01-20 mg/kg of the subject's body weight. For example, the dosage may be 0.3 mg/kg body weight, 1 mg/kg body weight, 3 mg/kg body weight, 5 mg/kg body weight, 10 mg/kg body weight or 20 mg/kg body weight, or within the range of 1-20 mg/kg body weight. Exemplary treatment regimens require administration once weekly, twice weekly, once every two weeks, once every three weeks, once every four weeks, once monthly, once every three months, once every 3-6 months, or with an initial shorter dosing interval followed by extended intervals. In an embodiment, the dosage used may be 1200 mg administered once every three weeks. The mode of administration may be intravenous infusion. In an embodiment, the administration may occur twice weekly. The administration mode may be intraperitoneal injection.

As used herein, “therapeutically effective dose” refers to a dose that reduces the severity of disease symptoms, increases the frequency and duration of asymptomatic periods, or prevents injury or disability caused by disease-related suffering. For example, a therapeutically effective dose is for anti-proliferative effects, preventing further tumor development, reducing tumor size, decreasing tumor vasculature, reducing cancer cell numbers, inhibiting, delaying, or reducing the growth and/or metastasis of tumors and/or malignant cells in cancer patients, and/or reducing one or more observable symptoms associated with the disease. The therapeutically effective dose may vary depending on numerous different factors, including the mode of administration, target site, physiological state of the patient, whether the patient is human or another animal, other medications administered, and whether the treatment is prophylactic or therapeutic. In certain embodiments, the patient is a human, but non-human mammals, including transgenic animals, may also be treated. Conventional methods known to those skilled in the art may be used to titrate the therapeutic dose to optimize safety and efficacy.

The “therapeutically effective dose” of the antibody or antigen-binding fragment thereof of the present invention will preferably inhibit cell growth or tumor growth by at least about 10%, preferably at least about 20%, more preferably at least about 30%, more preferably at least about 40%, more preferably at least about 50%, more preferably at least about 60%, more preferably at least about 70%, more preferably at least about 80%. The ability to inhibit tumor growth can be evaluated in animal model systems predictive of efficacy in human tumors. Alternatively, it may also be evaluated by examining the ability to inhibit cell growth, which can be determined in vitro by experiments well known to those skilled in the art. The effective amount of the antibody or antigen-binding fragment thereof of the present invention is capable of reducing tumor size or otherwise alleviating symptoms in the subject, such as preventing and/or treating metastasis or recurrence. Those skilled in the art can determine such amounts based on factors such as the size of the subject, the severity of the symptoms in the subject, and the specific composition or route of administration selected.

### Treatment

In yet another aspect, the present invention relates to use of the anti-CD100 antibody or antigen-binding fragment thereof, the pharmaceutical composition or the pharmaceutical combination of the present invention for the manufacture of a medicament for treating a disease in a subject.

The present invention also relates to the anti-CD100 antibody or antigen-binding fragment thereof, the pharmaceutical composition or the pharmaceutical combination of the present invention for use in treating a disease.

The present invention also provides a method for treating a disease in a subject, comprising administering to the subject a therapeutically effective amount of the anti-CD100 antibody or antigen-binding fragment thereof, the pharmaceutical composition, or the pharmaceutical combination of the present invention.

In an embodiment, the disease as described above is a cancer. The blocking of CD100 by the antibody of the present invention can enhance the immune response to cancer cells in patients. CD100 is widely expressed in many human tumors, and its expression is associated with human aggressive diseases. In preclinical tumor microenvironment, inflammatory cells and tumor cells express CD100 to regulate the infiltration, spatial distribution and activity of myeloid and lymphoid cells. CD100 binds to Plexin receptors located on myeloid cells in the tumor microenvironment. When the CD100 protein is blocked, the CD100 barrier can be eliminated. Once the barrier is breached, inflammatory dendritic cells and pro-inflammatory antigen-presenting cells migrate and infiltrate into the tumor. In preclinical cancer animal models, blocking CD100 with antibodies can delay tumor growth and promote durable tumor rejection.

As used herein, the “cancer” includes but is not limited to a hematological tumor or a solid tumor. Here, the solid tumor comprises, for example, squamous cell carcinoma, adenocarcinoma, basal cell carcinoma, renal cell carcinoma, breast ductal carcinoma, soft tissue sarcoma, osteosarcoma, melanoma, small cell lung cancer, non-small cell lung cancer, lung adenocarcinoma, peritoneal cancer, hepatocellular carcinoma, gastrointestinal cancer, gastric cancer, pancreatic cancer, neuroendocrine carcinoma, glioblastoma, cervical cancer, ovarian cancer, liver cancer, bladder cancer, brain cancer, hepatoma, breast cancer, colon cancer, colorectal cancer, endometrial cancer or uterine cancer, esophageal cancer, salivary gland cancer, kidney cancer, liver cancer, prostate cancer, vulvar cancer, thyroid cancer or head and neck cancer, etc., or any combination thereof. The hematological tumor comprises, for example, leukemia, lymphoma, myeloma, acute myeloid leukemia, chronic myeloid leukemia, acute lymphoblastic leukemia, chronic lymphocytic leukemia, hairy cell leukemia, Hodgkin's lymphoma, non-Hodgkin's lymphoma, or multiple myeloma, etc., or any combination thereof. The cancer can also be a metastatic cancer. “Metastasis” refers to the spread of cancer cells from their primary site to other parts of the body.

### Combination Therapy

For cancer treatment, the anti-CD100 antibody or antigen-binding fragment thereof, the pharmaceutical composition or the pharmaceutical combination of the present invention may be administered in combination with other therapeutic methods, including but not limited to: surgery, chemotherapy, radiation therapy, targeted therapy, immunotherapy, hormone therapy, angiogenesis inhibition, and palliative care.

The anti-CD100 antibody or antigen-binding fragment thereof, the pharmaceutical composition or the pharmaceutical combination of the present invention may further be administered in combination with at least one or more therapeutic agents described herein. The mode of co-administration is not limited. For example, the following therapeutic agents may all be administered in a single administration or separately. When administered separately (under different administration regimens), they may be administered continuously without interruption or at predetermined intervals.

In certain embodiments, the anti-CD100 antibody or antigen-binding fragment thereof, the pharmaceutical composition, or the pharmaceutical combination of the present invention is further administered in combination with one or more therapeutic agents selected from the group consisting of chemotherapeutic agents, radioisotopes, immune checkpoint inhibitors, and tumor antigen targeting drugs. Chemotherapeutic agents may include, for example, antimetabolites, alkylating agents, cytotoxic agents, topoisomerase inhibitors, microtubule inhibitors. Tumor antigen targeting drugs include, but are not limited to, agents that target tumor-associated antigens and tumor-specific antigens. Other non-limiting examples of therapeutic agents may include, for example, angiogenesis inhibitor, histone deacetylase (HDAC) inhibitors. Hedgehog signaling pathway blockers, mTOR inhibitors, p53/mdm2 inhibitors, PARP inhibitors, proteasome inhibitors (e.g., bortezomib, carfilzomib, ixazomib, Marizomib, Oprozomib), and tyrosine kinase inhibitors (e.g., BTK inhibitors).

In a preferred embodiment, the anti-CD 100 antibody A9-VHH, A31-VHH, A9, A31, A9-VHH1, A9-VHH2, A9-VHH3 or A31-VHH1 is used in combination with anti-PD-L1 antibody m18 or m18-VHH.

In a preferred embodiment, the anti-CD100 antibody A9-VHH1, A9-VHH2, A9-VHH3 or A31-VHH1 is administered in combination with the anti-PD-L1 antibody m18 or m18-VHH.

In a preferred embodiment, the anti-CD100 antibody A9-VHH2 is administered in combination with the anti-PD-L1 antibody m18 or m18-VHH.

In some embodiments, the anti-CD100 antibody or antigen-binding fragment thereof, the pharmaceutical composition or the pharmaceutical combination of the present invention is administered in combination with a chemotherapeutic agent. In some embodiments, the anti-CD100 antibody or antigen-binding fragment thereof, the pharmaceutical composition or the pharmaceutical combination of the present invention is administered in combination with an immune checkpoint inhibitor. In some embodiments, the anti-CD100 antibody or antigen-binding fragment thereof, the pharmaceutical composition or the pharmaceutical combination of the present invention is administered in combination with a radioisotope. In some embodiments, the anti-CD100 antibody or antigen-binding fragment thereof, the pharmaceutical composition or the pharmaceutical combination of the present invention is administered in combination with a tumor antigen targeting drug.

### Kit

The present invention further provides a kit comprising the anti-CD100 antibody or antigen-binding fragment thereof, the pharmaceutical composition or the pharmaceutical combination of the present invention, and instructions for use. The kit may further comprise a suitable container. In certain embodiments, the kit additionally comprises a device for administration. The kit generally includes a label indicating the intended use and/or method of use of the kit contents. The term “label” includes any written or recorded material provided on or with the kit, or otherwise accompanying the kit.

In an embodiment, the kit comprises the anti-CD100 antibody A9-VHH, A31-VHH, A9, A31, A9-VHH1, A9-VHH2, A9-VHH3, A31-VHH1, or a combination thereof.

In an embodiment, the kit comprises the anti-CD100 antibody A9-VHH1, A9-VHH2, A9-VHH3, A31-VHH1, or a combination thereof.

In an embodiment, the kit comprises the anti-CD100 antibody A9-VHH2.

In an embodiment, the kit comprises a combination of the anti-CD 100 antibody A9-VHH, A31-VHH, A9, A31, A9-VHH1, A9-VHH2, A9-VHH3 or A31-VHH1 and the anti-PD-L1 antibody m18 or m18-VHH.

In an embodiment, the kit comprises a combination of the anti-CD100 antibody A9-VHH1, A9-VHH2, A9-VHH3or A31-VHH1 and the anti-PD-L1 antibody m18 or m18-VHH.

In an embodiment, the kit comprises a combination of the anti-CD100 antibody A9-VHH2 and the anti-PD-L1 antibody m18 or m18-VHH.

### Beneficial Effects

The anti-CD100 antibody or antigen-binding fragment thereof according to the present invention has at least one of the following beneficial effects: 1) having affinity for human, mouse or monkey CD100 protein; 2) having affinity for CD100-positive cells; 3) blocking the binding of CD100 to Plexin-B1 or Plexin-B2; 4) inhibiting the proliferation of MDSC cells; and 5) inhibiting tumor growth.

The combination of the anti-CD100 antibody or antigen-binding fragment thereof according to the present invention with the anti-PD-L1 antibody or antigen-binding fragment thereof as described herein has at least one of the following beneficial effects: 1) inhibiting or delaying tumor growth; 2) significantly increasing the response rate to monotherapy; 3) prolonging survival; 4) enhancing the therapeutic effect of PD-L1 tumor immunotherapy; and 5) high safety profile.

### EXAMPLES

Further understanding of the present invention can be obtained by reference to some specific examples provided herein, which are intended merely to illustrate the invention and are not intended to pose a limitation on the scope of the invention. It will be apparent that various modifications and variations can be made to the invention without departing from its spirit, and therefore, such modifications and variations also fall within the scope claimed in the present application. The proportions used herein include percentages. Unless otherwise specified, all percentages mentioned herein are on a weight basis.

### Example 1 Preparation and Identification of Materials

### 1. 1 Preparation and Identification of CD100 Control Antibody

Preparation of CD100 control antibody: anti-CD100 antibody Pepinemab was used as a positive control antibody. The gene sequence encoding the Pepinemab monoclonal antibody, as disclosed in WO2013148854A1, was synthesized by General Biosystems Co., Ltd. It was then constructed into the eukaryotic expression vector pcDNA3.4 (Invitrogen) by homologous recombination. The constructed recombinant protein expression vector was transformed into Escherichia coli DH5α, cultured overnight at 37° C., and then subjected to plasmid extraction using an endotoxin-free plasmid extraction kit (OMEGA, D6950-01) to obtain the desired expression plasmid expressing Pepinemab monoclonal antibody. The expression plasmid was transfected into CHO cells using ExpiFectamine™ CHO Transfection Kit (Thermo Fisher, A29129) according to the manufacturer's instructions to express the Pepinemab monoclonal antibody. 7 days after transfection, the cell culture supernatant was collected and centrifuged at 15,000 g for 10 min. After the resulting supernatant was filtered through a 0.22 μm membrane, the antibody in the supernatant was affinity-purified using a Protein A/G affinity chromatography column (MabSelect SuRe (Cytiva, 17543802)). The target antibody was eluted with 100 mM glycine buffer (pH 3.0) and the eluted antibody was exchanged into PBS buffer using an ultrafiltration concentration tube (Millipore, UFC901096).

Identification of CD100 control antibody: The activity of the prepared positive control antibody Pepinemab (heavy chain constant region type is IgG4SP) was detected by using the purchased Human Semaphorin 4D/SEMA4D/CD100 Protein, Fc Tag antigen protein (Acro, CD0-H5257), and the specific procedures were as follows: the 96-well ELISA plate was coated with the purchased human CD100 protein (also referred to as HuCD100-Fc, 2 μg/mL, 30 μL/well), and coated overnight at 4° C.; after washing the plate 3 times, it was blocked with 5% skim milk prepared in PBS at room temperature for 1 hour; after washing the plate 3 times, the serially diluted control antibody Pepinemab in PBS was added and the plate was incubated at room temperature for 1 hour; after washing the plate, the secondary antibodies Anti-human-IgG-Kappa-HRP (Millipore, AP502P) and Anti-human-IgG-Lambda-HRP (Millipore, AP506P) diluted in PBS (1:6000) were added and the plate was incubated at room temperature for 1 hour. The plate was washed 6 times, and TMB (SurModics, TMBS-1000-01) was added for color development (5-20) minutes). After the color reaction was terminated, data were read at OD450) using a microplate reader (Molecular Devices, SpecterMax 190). Data were processed and plotted using Graphpad Prism. The results, as shown in FIG. 1. indicated that the expressed control antibody Pepinemab bound to the CD100 protein and exhibited normal anti-CD100 activity.

### 1.2 Preparation and Identification of CD100 Antigen Protein

Preparation of antigen protein: through genetic manipulation at the coding gene level, human Fc or His tags was added to the C-terminus of the amino acid sequences of human CD100 protein ECD region fragment from position 22 to 734 (HuCD100, Uniprot ID:Q92854), mouse CD100 protein ECD region fragment from position 24 to 733 (MusCD100, Uniprot ID:O09126), and cynomolgus monkey CD100 protein ECD region fragment from position 22 to 734 (CynoCD100, Uniprot ID:A0A2K5TZC9). The obtained nucleic acid sequences were constructed into the pcDNA3.4 vector, then transformed into Escherichia coli DH5α and cultured overnight at 37° C., respectively. Subsequently, plasmids were extracted using an endotoxin-free plasmid extraction kit (OMEGA, D6950-01). The resulting plasmids were transiently transfected into HEK293 cells (ATCC® CRL-1573™) using the ExpiFectamine™ 293 Transfection Kit (Gibco™, A14524). 7 days after expression, cell culture supernatants were collected. For proteins with an Fc tag, after cell culture, the cell suspension was centrifuged at high speed to collect the supernatant, and the resulting supernatant was filtered through a 0.22 um membrane and purified via affinity chromatography using a Protein A/G column MabSelect SuRe (Cytiva, 17543802). The target protein was eluted with 100 mM glycine hydrochloride (pH 3.0), followed by concentration, buffer exchange, aliquoting, SDS-PAGE identification, and activity detection before it was stored frozen. For proteins with a His tag, affinity purification was performed using Ni Smart Beads 6FF (Changzhou Smart-Lifesciences Biotechnology Co., Ltd., SA036050), and the target protein was eluted via an imidazole gradient. Each eluted protein was exchanged into PBS buffer using an ultrafiltration concentration tube (Millipore, UFC901096). Finally, human, mouse, and monkey CD100 antigen proteins with Fc tags and His tags (HuCD100-Fc, MusCD100-Fc, CynoCD100-Fc, HuCD100-His, MusCD100-His, CynoCD100-His) were obtained.

Antigen identification: the quality-validated antibody Pepinemab (IgG4SP) obtained in Example 1.1 was used to assess the prepared antigens (HuCD100-Fc, MusCD100-Fc, CynoCD100-Fc, HuCD100-His, MusCD100-His, CynoCD100-His).

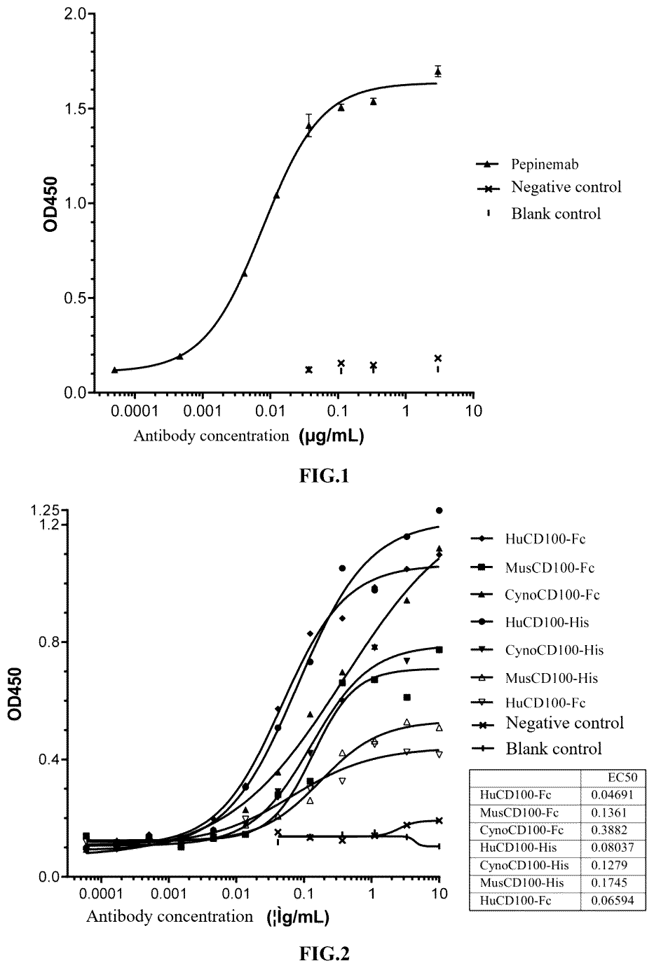

The specific procedures were as follows: the ELISA plate was coated with 2 μg/mL antigen at 4° C. overnight, and the purchased antigen protein Human Semaphorin 4D/SEMA4D/CD100 Protein, Fc Tag antigen protein (Acro, CDO-H5257) was used as a positive control; after washing the plate 3 times, it was blocked with 5% skim milk prepared in PBS at room temperature for 1 hour; after washing the plate 3 times, the serially diluted antibody Pepinemab in PBS was added and the plate was incubated at room temperature for 1 hour; after washing the plate, the secondary antibody Anti-human-IgG-Kappa-HRP (Millipore, AP502P) or Anti-human-IgG-Lambda-HRP (Millipore, AP506P) diluted in PBS (1:6000) was added and the plate was incubated at room temperature for 1 hour. The plate was washed 6 times, and then TMB (SurModics, TMBS-1000-01) was added for color development (5-20) minutes). After the color reaction was terminated, data were read at OD450) using a microplate reader. Data were processed and plotted using Graphpad Prism. The results, as shown in FIG. 2. indicated that the binding affinity of the antibody Pepinemab to the in-house prepared antigen was comparable to that to the purchased CD100 antigen protein. The two HuCD100-Fc, listed in order, were the in-house prepared HuCD100 antigen and the commercially available HuCD100 antigen.

### Example 2 Construction and Identification of Overexpression Cell Lines

### 2.1 Construction and Identification of CD100 Overexpressing Cell Lines

Construction of HEK293 cell lines overexpressing CD 100 (hereinafter referred to as HuCD100-HEK293, MusCD100-HEK293, CynoCD100-HEK293): the coding nucleic acid sequences of full-length human CD100 protein HuCD100 (Uniprot ID: Q92854), full-length mouse CD100 protein MusCD100 (Uniprot ID: O09126), and full-length cynomolgus monkey CD100 protein CynoCD100 (Uniprot ID: A0A2K5TZC9) were respectively constructed into the pLVX-puro plasmid (Clontech, 632164). The resulting plasmids were then electroporated into HEK293 cells (ATCCR CRL-1573TM) using an electroporator (Invitrogen, Neon™ Transfection System, MP922947). After electroporation, the obtained cells were transferred into DMEM medium (Gibco, 11995065) containing 10% (v/v) FBS (Gibco, 15140-141) without antibiotics. The cells were then transferred to 10×10 cm cell culture dishes and cultured for 48 hours followed by being seeded into 96-well cell culture plates at a density of 10⁴ cells/well. Puromycin at a final concentration of 2 μg/mL was added as a selection pressure, and cell lines forming clones were picked for identification after approximately 2 weeks.

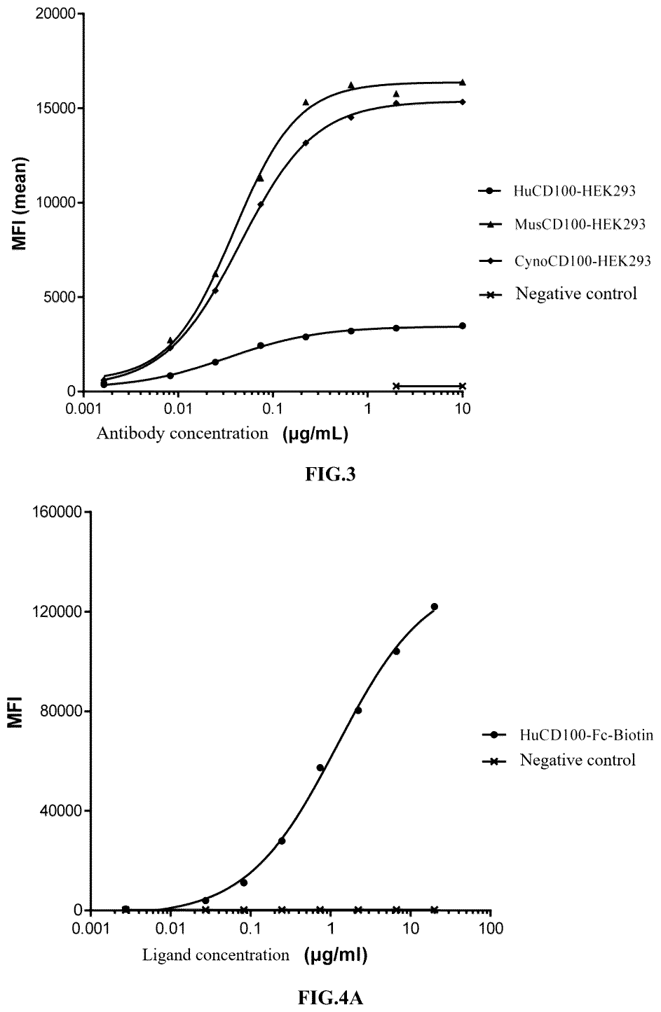

Flow cytometry identification of HuCD100-HEK293, MusCD100-HEK293, and CynoCD100-HEK293 cells: cells of the above cell lines in the logarithmic growth phase were trypsinized and plated into 96-well plates. After washing with FACS buffer (1×PBS buffer containing 2% (v/v) FBS), the serially diluted primary antibody (Pepinemab) in PBS was added and the plates were incubated at 4° C. for 30 min. After washing, the prepared fluorescent secondary antibody anti human IgG Fc (abcam, 98596) was added and the plates were incubated at 4° C. for 30 min. Finally, detection was performed using a flow cytometer (Beckman, CytoFLEXAOO-1-1102). The detection results, as shown in FIG. 3, indicated that HuCD100-HEK293, MusCD100-HEK293, and CynoCD100-HEK293 cell lines highly expressed human, mouse, and monkey CD100 on their surfaces, respectively.

### 2.2 Construction and Identification of Cell Lines Overexpressing HuPlexin-B1, MusPlexin-B1, CynoPlexin-B1, and HuPlexin-B2

Construction of HEK293 Cell lines overexpressing Plexin-B1 and Plexin-B2 (hereinafter referred to as HuPlexin-B1-HEK293, MusPlexin-B1-HEK293, CynoPlexin-B1-HEK293, and HuPlexin-B2-HEK293): the coding nucleic acid sequences of full-length human Plexin-B1 protein HuPlexin-B1 (Uniprot ID: 043157), full-length mouse Plexin-B1 protein MusPlexin-B1 (Uniprot ID: Q8CJH3), full-length cynomolgus monkey Plexin-B1 protein CynoPlexin-B1 (Uniprot ID: A0AID5QMB8), and full-length human Plexin-B2 protein HuPlexin-B2 (Uniprot ID: 015031) were constructed into the pLVX-puro plasmid (Clontech, 632164). For the construction procedures, see Example 2.1.

Flow cytometry identification of HuPlexin-B1-HEK293, MusPlexin-B1-HEK293, CynoPlexin-B1-HEK293, and HuPlexin-B2-HEK293 cells: cells of the above cell lines in the logarithmic growth phase were trypsinized and plated into 96-well plates. After washing with FACS buffer (1×PBS buffer containing 2% (v/v) FBS), the serially diluted antigen proteins (HuCD100-Fc-Biotin, MusCD100-Fc-Biotin, CynoCD100-Fc-Biotin) in PBS were added and the plates were incubated at 4° C. for 30 min. After washing, the prepared eBioscience Streptavidin PE (Invitrogen, 2265658, 1:300) was added and the plates were incubated at 4° C. for 30 min. Finally, detection was performed using a flow cytometer (Beckman, CytoFLEXAOO-1-1102). The detection results, as shown in FIGS. 4A-4D, indicated that HuPlexin-B1-HEK293, MusPlexin-B1-HEK293, CynoPlexin-B1-HEK293, and HuPlexin-B2-HEK293 cell lines highly expressed human, mouse, and monkey Plexin-B1 and human Plexin-B2 on their surfaces, respectively.

### Example 3 Animal Immunization and Construction of Immune Library

### 3.1 Immunization Protocol

In this example, a cross-immunization protocol using HuCD100-Fc and HuCD100-His antigens was employed via subcutaneous injection in an alpaca (Shenzhen Kangti Life Technology Co., Ltd.). Immunization was performed every two weeks for a total of 4 doses. Alpaca blood was collected one week after the 4th immunization for immune titer detection.

### 3.2 Detection of Antibody Titers in Alpaca Serum After Immunization

The ELISA plates were coated overnight at 4° C. with 2 μg/mL HuCD100-Fc, HuCD100-His, MusCD100-Fc, MusCD100-His, CynoCD100-Fc, and CynoCD100-His, respectively. After washing the plates 3 times, they were blocked with 5% skim milk prepared in PBS at room temperature for 1 hour. After washing the plates 3 times, the serially diluted alpaca serum in PBS was added, with antibody Pepinemab included as a positive control, and the plates were incubated at room temperature for 1 hour. After washing the plates, the secondary antibody Goat anti-Llama IgG (H+L) Secondary Antibody [HRP] (NOVUSBIO, NB7242) or Anti VHH-HRP 1641&CP001 (Genescript, A02089/A01861-200) diluted in PBS was added and the plates were incubated at room temperature for 1 hour. After washing the plates 6 times, TMB was added for color development (5-20) minutes). Following termination of the color reaction, data were read at OD450 μsing a microplate reader. The results showed that serum antibody titers were still detectable at a serum dilution of 1:128 k, indicating all alpaca serum titers met the standard (data not shown).

### 3.3 Construction of Phage Display Antibody Gene Library

After animal immunization, 80 mL of alpaca blood was collected, and PBMCs were isolated using Ficoll-Paque density gradient media (GE, 17144003S) for construction of the alpaca immune library. For specific procedures, see Examples 2 and 3 of patent CN111978402A.

### 3.4 Screening of Phage Display Antibody Gene Library

In this example, a phage display antibody gene library was constructed and screened using the antigen proteins HuCD100-Fc, MusCD100-Fc, CynoCD100-Fc, HuCD100-His, MusCD100-His, and CynoCD100-His prepared in Example 1.2 as screening antigens, and multiple antibody molecules specifically binding to human CD100 were obtained.

### 3.4.1 Screening of Phage Display Antibody Gene Library by Magnetic Bead Method

The magnetic bead screening method is a panning process that antigen proteins were biotinylated and then bound to streptavidin-conjugated magnetic beads, and the antigen-bound beads were incubated with the phage display antibody gene library, followed by washing and elution. Typically, 3-4 rounds of panning were performed to enrich specific monoclonal antibodies against the antigen. In this example, biotinylated antigen proteins HuCD100-Fc, CynoCD100-Fc, HuCD100-His, and MusCD100-His were used for phage display library screening. After 3 rounds of panning, primary screening of monoclonal antibody VHH against human CD100 was conducted. For specific procedures, see Example 2.4.1 of CN112250763A.

### 3.4.2 Screening of Phage Display Antibody Gene Library by Immunotube Screening Method

The immunotube screening method is a panning process that antigen proteins HuCD100-Fc, CynoCD100-Fc, HuCD100-His, and MusCD100-His were coated on the surface of high-adsorption immunotubes, the phage display antibody library was added to the immunotubes and incubated with the immunotube surface-adsorbed antigens, followed by washing and elution. After 2-4 rounds of panning, specific monoclonal antibodies VHHs against the antigen were finally enriched. In this example, the monoclonal antibodies VHHs against human CD100 were enriched after 3 rounds of panning. For specific procedures, see Example 2.4.2 of CN112250763A.

3.5 Selection of Monoclonal Clones To evaluate the enrichment efficiency, ELISA was performed on the phage pools eluted in each round. Ten clones were randomly selected from each round's phage pool for sequence analysis. Based on a comprehensive analysis of the enrichment efficiency and the redundancy rates of the tested sequence, the appropriate round was selected for monoclonal clone picking.

For primary ELISA screening of monoclonal clones, antigen proteins HuCD100-His, MusCD100-His, and CynoCD100-His were used. The VHH antibodies obtained from primary screening that bound to HuCD100-His, MusCD100-His, and CynoCD100-His were prepared as VHH lysates. These were then further verified by flow cytometry (FACS) using the overexpressing cells HuCD100-HEK293, MusCD100-HEK293, and CynoCD100-HEK293 prepared in Example 2.1. 2 antibody VHH molecules specifically binding to human CD100 were screened, and named with their respective clone numbers (A9-VHH, A31-VHH). The amino acid sequences of the CDR regions of the obtained antibody VHHs are shown in Table 1, with CDR sequences determined using the AbM definition.

TABLE 1
Amino acid sequences of variable regions of
two anti-CD100 VHH antibodies (SEQ ID NO:)

| Antibody Name | CDR1 | CDR2 | CDR3 | VHH |
|---|---|---|---|---|
| A9-VHH | 1 | 2 | 3 | 4 |
| A31-VHH | 5 | 6 | 7 | 8 |

### Example 4 Antibody Construction, Expression and Purification

### 4.1 Plasmid Construction

Chimeric antibodies were designed based on the candidate nanobodies screened in Example 3. When constructing the candidate nanobody genes into the transient expression plasmid pcDNA3.4 (Thermofisher, A14697), a human IgG Fc fragment was fused to the C-terminus of the candidate nanobodies. Specifically, a human IgG4SP Fc fragment (SEQ ID NO: 19) was fused to the C-terminus of the candidate monoclonal nanobodies A9-VHH and A31-VHH obtained from screening. The constructs were inserted into the eukaryotic expression vector plasmid pcDNA3.4 (Invitrogen) and transformed into Escherichia coli DH5α, followed by overnight culture at 37° C. The plasmids were extracted by using an endotoxin-free plasmid extraction kit (OMEGA, D6950-01), and the endotoxin-free antibody plasmids were obtained for eukaryotic expression. The two designed VHH-Fc chimeric antibodies were named A9 and A31, respectively. The amino acid sequences of the designed chimeric antibodies are shown in Table 2.

TABLE 2
Amino acid sequences of 2 anti-CD100 antibodies

| Antibody Name | SEQ ID NO: |
|---|---|
| A9 | 9 |
| A31 | 10 |

### 4.2 Antibody Expression and Purification

The antibody sequences obtained above were expressed using the ExpiCHO transient expression system kit (Thermo Fisher, A29133) according to the ExpiCHO™ Expression System USER GUIDE. The specific procedures were as follows: on the day of transfection, when confirming that the CHO cell density was approximately 7×10⁶-1×10⁷ viable cells/mL and the cell viability was >98%, the cells were adjusted to a density of 6×10⁶ cells/mL with pre-warmed (37° C.) fresh ExpiCHO expression medium. The target plasmid was diluted with pre-chilled (4° C.) OptiPRO™ SFM (1 μg plasmid was added to 1 mL OptiPRO™ SFM), and the ExpiFectamine™ CHO reagent was simultaneously diluted with OptiPRO™ SFM. The two solutions were then mixed in equal volumes and gently pipetted to prepare an ExpiFectamine™ CHO/plasmid DNA mixture. The mixture was incubated at room temperature for 1-5 min, then slowly added to the prepared cell suspension with gently shaking, and finally, the cell culture was placed on a cell culture shaker at 37° C. with 8% CO₂.

18-22 h after transfection, ExpiCHO™ Enhancer reagent and ExpiCHO™ Feed reagent were added to the cell culture medium, and the flasks were placed on a shaker at 32° C. with 5% CO₂ for continued culture. On day 5 after transfection, an equal volume of ExpiCHO™ Feed reagent was added, gently mixing the cell suspension while adding slowly. 7 days after transfection, the cell culture supernatant containing the target antibody protein was collected and centrifuged at 15,000 g for 10 min. The resulting supernatant was affinity-purified using MabSelect SuRe LX (GE, 17547403), and the target antibody protein was eluted with 100 mM sodium acetate (pH 3.0), neutralized with 1 M Tris-HCl, and finally exchanged into PBS buffer using an ultrafiltration concentration tube (Millipore, UFC901096).

### Example 5 Physicochemical Property Detection of Antibodies

In this example, SDS-PAGE and SEC-HPLC were used to detect the relative molecular weight and purity of candidate antibodies.

### 5.1 Identification of Antibodies by SDS-PAGE

Preparation of non-reduced solution: 1 μg of each obtained antibody and the quality control product IPI (Ipilimumab, for example, prepared as described in WO2001014424) were added to 5×SDS loading buffer and 40 mM iodoacetamide, followed by dry bath heating at 75° C. for 10 min. After cooling the mixture to room temperature, they were centrifuged at 12,000 rpm for 5 min, and the supernatants were collected.

Preparation of reduced solution: 2 μg of each obtained antibody and the quality control product IPI were added to 5×SDS loading buffer and 5 mM DTT, followed by dry bath heating at 100° C. for 10 min. After cooling the mixture to room temperature, they were centrifuged at 12,000 rpm for 5 min, and the supernatants were collected. The supernatant was loaded onto a Bis-tris 4-15% gradient gel (GenScript) for gel electrophoresis, and protein bands were stained by Coomassie Brilliant Blue staining.

The protein gel with visualized protein bands (after destaining the gel with destaining solution until the background was transparent) was scanned using an EPSON V550 color scanner. The purity of reduced and non-reduced bands was calculated by peak area normalization method using ImageJ.

Experimental results showed that the bands of each antibody on the non-reduced gel were approximately 80 kD, and those on the reduced gel were approximately 40 kD, consistent with the expected sizes. The results, as shown in Table 3, indicated that all candidate antibodies detected by reduced gel exhibited a purity of more than 95%.

### 5.2 Identification of Antibody Monomeric Purity by SEC-HPLC

Material preparation: mobile phase: 150 mmol/L phosphate buffer, pH 7.4; each antibody and quality control product IPI were diluted to 0.5 mg/mL with the mobile phase solution.

Experimental procedures: the Agilent HPLC 1100 or Shimadzu LC2030C PLUS liquid chromatograph was used, with an XBridge BEH chromatography column (SEC 3.5 μm, 7.8 mm I.D.×30 cm, Waters). The flow rate was set at 0.8 mL/min, the injection volume was 20 μL, and the VWD detector wavelengths were 280 nm and 214 nm. Blank solution, IPI quality control solution, and antibody sample solutions were injected in sequence.

The percentages of high-molecular-weight polymers, antibody monomers, and low-molecular-weight substances in the samples were calculated by the area normalization method. The results, as shown in Table 3, indicated that the monomeric purity of all candidate antibodies was more than 95%.

TABLE 3
Expression levels and physicochemical
properties of the candidate antibodies

| | | Expression | Expression | SDS- | SEC- |
|---|---|---|---|---|---|
| Antibody | | Volume | Level | PAGE | HPLC |
| Name | Species | (mL) | (μg/mL) | (%) | (%) |
| A9 | Alpaca | 10 | 11.90 | >95.0 | 98.91 |
| A31 | Alpaca | 10 | 43.50 | >95.0 | 100.00 |

### Example 6 Detection of Antigen-Binding Activity of Antibodies

In this example, the binding of expressed candidate antibodies (A9 and A31) to CD100 antigen proteins HuCD100-His, MusCD100-His, and CynoCD100-His was detected by ELISA. Additionally, the binding ability of the expressed antibodies (A9 and A31) to CD100-overexpressing cells (HuCD100-HEK293, MusCD100-HEK293, CynoCD100-HEK293), human peripheral blood mononuclear cells (hereafter referred to as HuPBMCs) naturally expressing human CD100, and human T lymphocyte leukemia Jurkat cells naturally expressing human CD100 was evaluated by FACS.

### 6.1 ELISA-Based Detection of Antibody Binding to CD100-His Antigen Protein

96-well ELISA plates were coated with 2 μg/mL HuCD100-His, MusCD100-His, and CynoCD100-His (30 μL/well) overnight at 4° C. The next day, the plates were washed 3 times with PBST and blocked with 5% skim milk for 2 hours. After another 3 washes with PBST, serially diluted antibodies and the positive control antibody Pepinemab were added and the plates were incubated for 1 hour. Following 3 additional washes with PBST, the secondary antibody Goat-anti-human Fc-HRP (abcam, ab97225) was added and the plates were incubated for 1 hour. After incubation, the plates were washed 6 times with PBST, and TMB (SurModics, TMBS-1000-01) was added for color development. According to the color development result, the reaction was terminated with 2 M stop solution, and absorbance was read at OD450 μsing a microplate reader (Molecular Devices, SpecterMax 190).

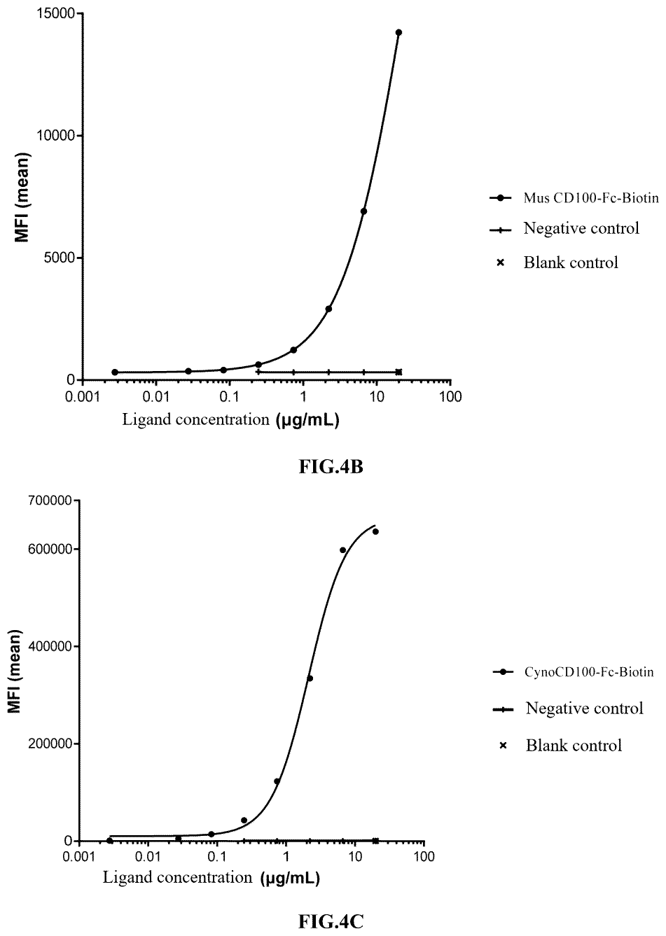

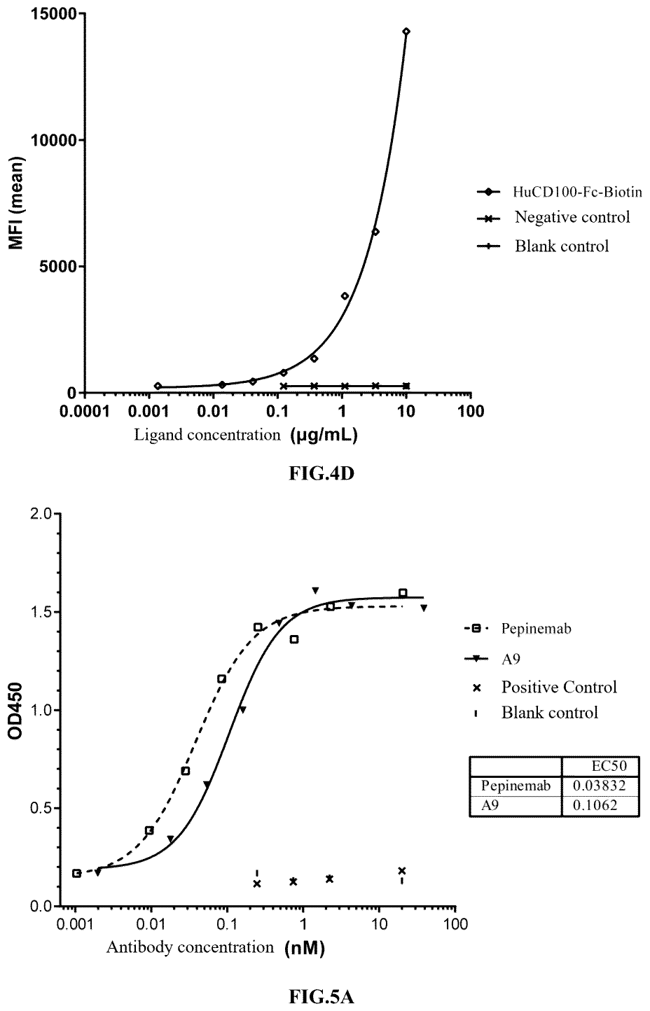

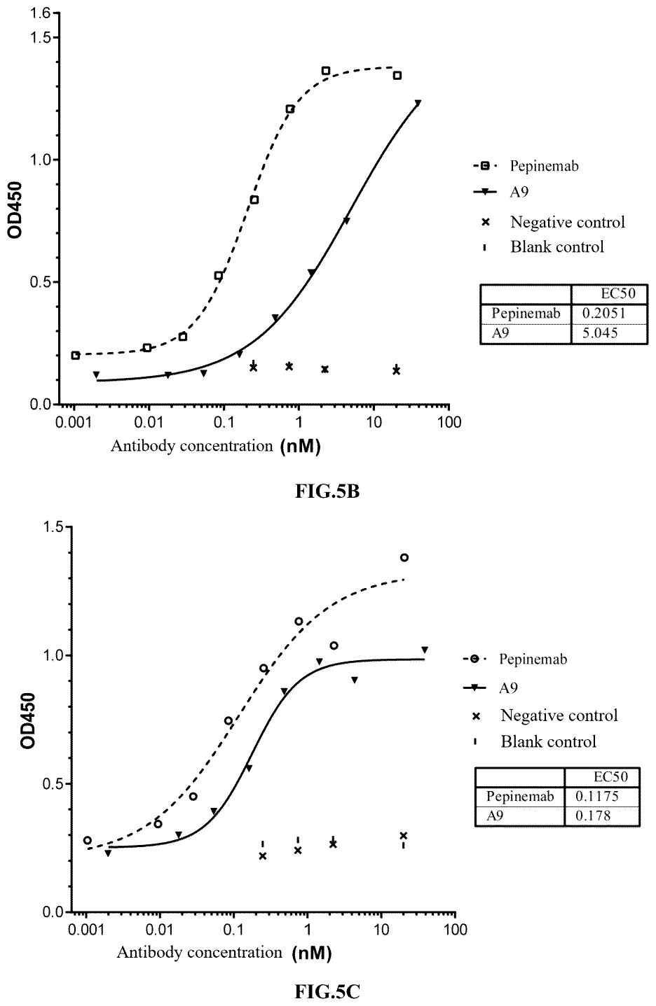

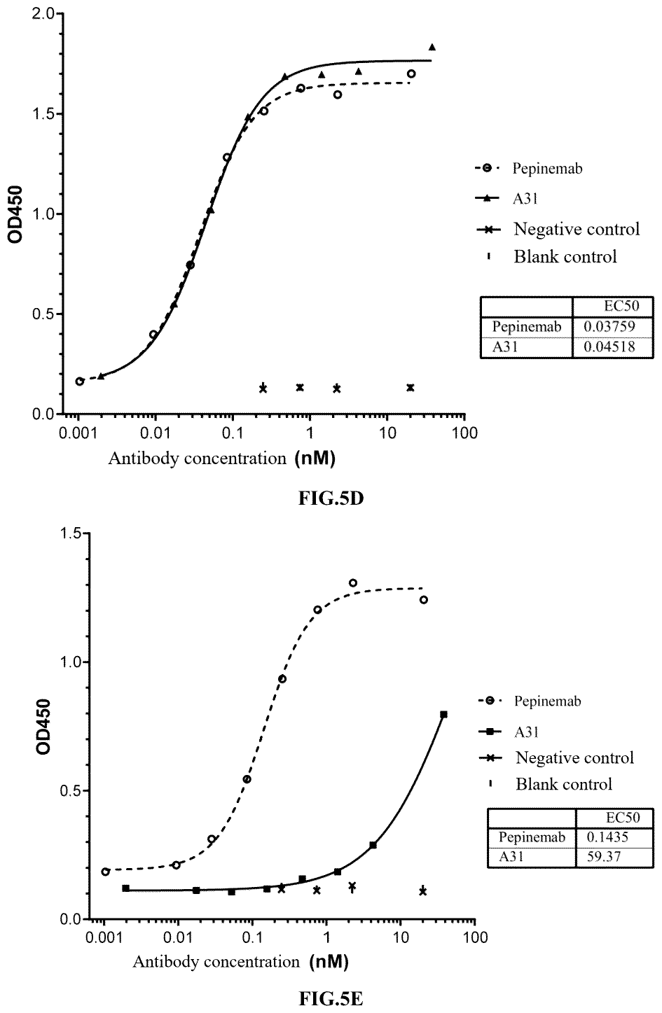

The results, as shown in FIGS. 5A-5F, indicated that the antibody molecule A9 exhibited good binding activity to HuCD100-His, MusCD100-His, and CynoCD100-His antigen proteins, although the binding activity was slightly lower than that of the positive control antibody against the corresponding antigens proteins. The antibody molecule A31 showed good binding activity to both HuCD100-His and CynoCD100-His and weaker binding activity to MusCD100-His. Moreover, the binding activity of A31 to CynoCD100-His is superior to that of the positive control antibody, while the binding activity of A31 to HuCD100-His is comparable to that of the positive control antibody.

### 6.2 FACS-Based Detection of Antibody Binding Ability to CD100-HEK293 Cells

In this example, the binding activity of antibodies was evaluated using human CD100-overexpressing cells (HuCD100-HEK293, MusCD100-HEK293, CynoCD100-HEK293).

The specific procedures were as follows: HuCD100-HEK293, MusCD100-HEK293, and CynoCD100-HEK293 cells in logarithmic growth phase were prepared as single-cell suspensions. The density was adjusted to 1×10⁶ cells/mL, and 100 μL of each suspension was added per well to 96-well plates. The plates were centrifuged at 4° C., 300 g and the supernatants were removed. The serially diluted antibodies and positive control antibody Pepinemab were added to corresponding wells, and the plates were mixed and incubated at 4° C. for 30 min. After incubation, the cell mixture was washed three times, and then 100 μL of 1:300 diluted secondary antibody Goat F(ab′)₂ Anti-Human IgG-Fc (PE) (abcam, ab98596) was added, and the plates were incubated in the dark at 4° C. for 30 min. Following 3 additional washes, detection was performed using a flow cytometer (Beckman, CytoFLEX AO0-1-1102).

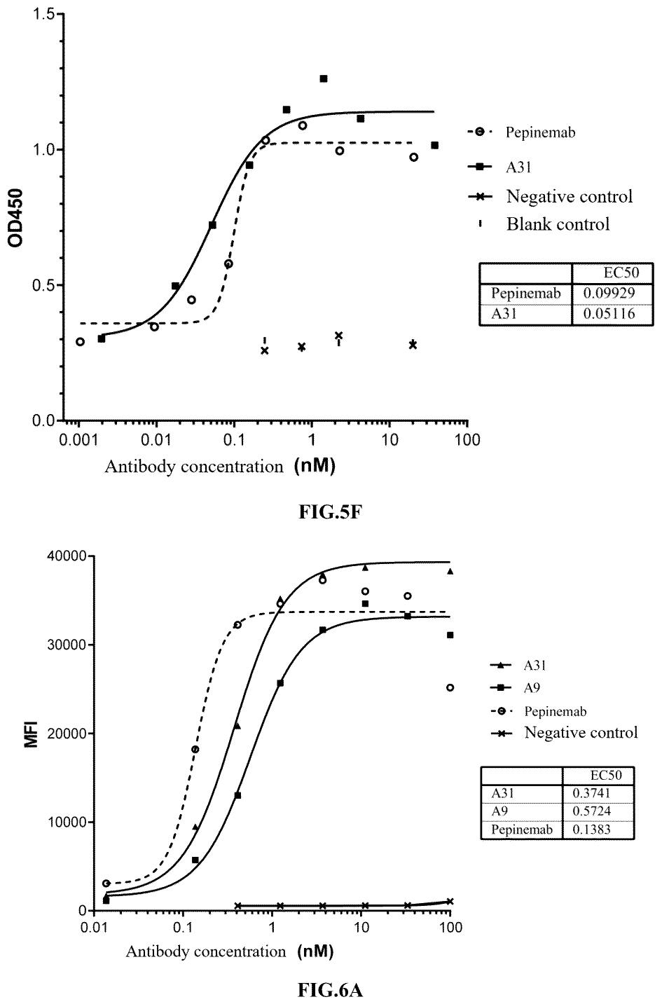

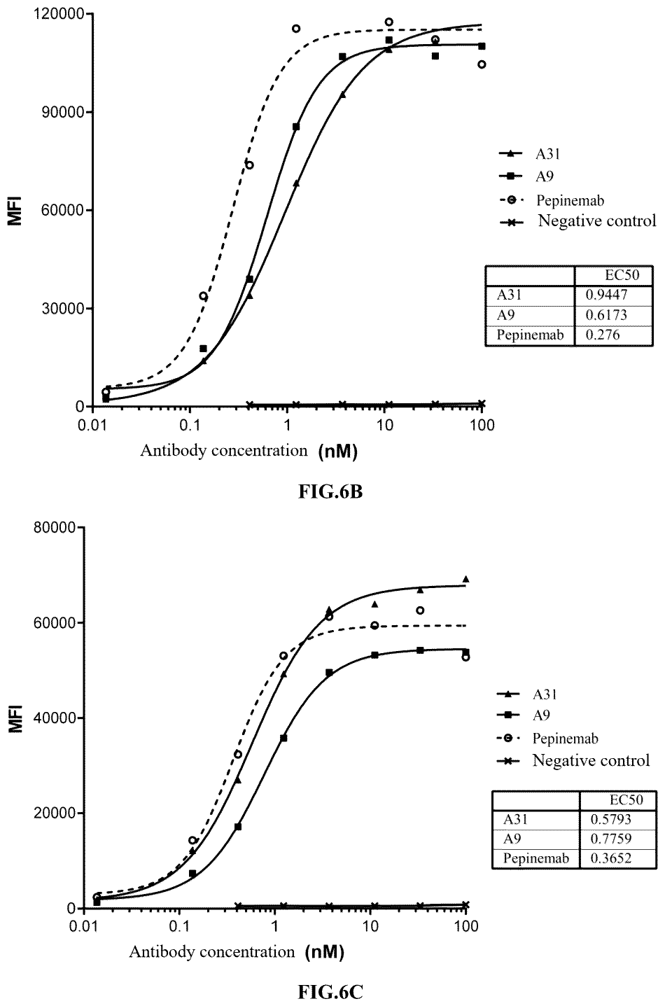

The results, as shown in FIGS. 6A-6C, indicated that both antibody molecules A9 and A31 exhibited good binding activity to HuCD100-HEK293, MusCD100-HEK293, and CynoCD100-HEK293 cells, although their binding activity was slightly lower than that of the positive control antibody against the respective overexpressing cell lines.

### 6.3 FACS-Based Detection of Antibody Binding Activity to HuPBMC and Jurkat Cells

In this example, the binding activity of antibodies was evaluated using human peripheral blood mononuclear cells (hereinafter referred to as HuPBMCs) and human T lymphocyte leukemia Jurkat cells (hereinafter referred to as Jurkat cells).

The specific procedures were as follows: HuPBMCs (AllCells, PB004F-C (Y1246)) and Jurkat cells (ATCC, TIB-152) in logarithmic growth phase were prepared as single-cell suspensions. The density was adjusted to 1×10⁶ cells/mL, and 100 μL of each suspension was added per well to 96-well plates. The plates were centrifuged at 4° C., 300 g and the supernatants were removed. The serially diluted antibodies and positive control antibody Pepinemab were added to corresponding wells, and the plates were mixed and incubated at 4° C. for 30 min. After incubation, the cell mixture was washed three times, and then 100 μL of 1:300 diluted secondary antibody Goat F(ab′)₂ Anti-Human IgG-Fc (PE) (abcam, ab98596) was added, and the plates were incubated in the dark at 4° C. for 30 min. Following 3 additional washes, detection was performed using a flow cytometer (Beckman, CytoFLEX AOO-1-1102).

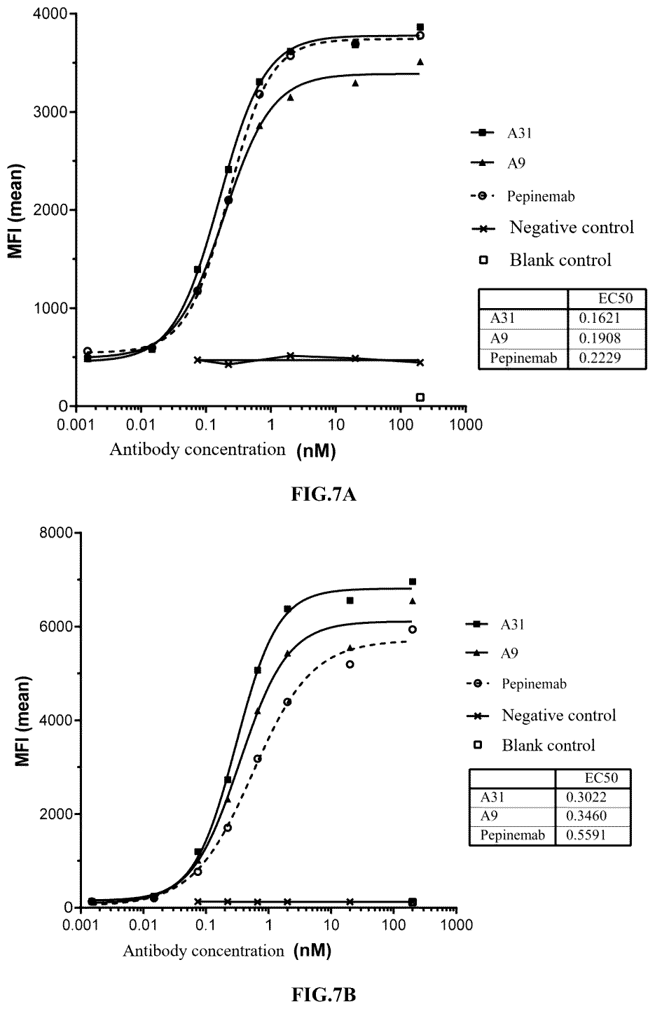

The results, as shown in FIGS. 7A-7B, indicated that, on Jurkat cells (FIG. 7B), both antibody molecules A9 (EC₅₀=0.346 nM) and A31 (EC₅₀=0.3022 nM) exhibited superior binding activity compared to the positive control antibody (EC₅₀=0.5591 nM). On HuPBMCs (FIG. 7A), the binding activity of the antibody molecules A31 (EC₅₀=0.1621 nM) and A9 (EC₅₀=0.1908 nM) to the cells were comparable to that of the positive control antibody (EC₅₀=0.2229 nM).

### Example 7 Blocking Activity Detection of Antibodies

In this example, the blocking activity of the candidate antibodies (A9 and A31) was evaluated using two cell lines: HuPlexin-B1-HEK293 and HuPlexin-B2-HEK293.

The specific procedures were as follows: cultured HuPlexin-B1-HEK293 and HuPlexin-B2-HEK293 cells were collected, centrifuged at 300 g to remove the supernatant, resuspended in prepared FACS buffer, counted, and adjusted to a cell suspension density of 1× 10⁶ cells/mL. HuPlexin-B1-HEK293 and HuPlexin-B2-HEK293 cells were added to 96-well plates at 100 μL per well. The plates were centrifuged at 300 g to remove the supernatant. The serially diluted antibodies and the positive control antibody Pepinemab were added to the corresponding wells, and the cells were resuspended, and incubated at 4° C. for 30 min. The diluted antibodies were mixed with 100 μL of biotinylated antigen protein HuCD100-Fc dilution (0.5 μg/mL) or CynoCD100-Fc dilution (0.3 μg/mL) (prepared in Example 1.1), and the mixture was added to the cells for co-incubation for 1 hour. The cell mixture was then washed 3 times, resuspended, and incubated at 4° C. for 30 min. After 3 additional washes, PE-conjugated streptavidin (eBioscience, 12-4317-87) was added, and the plates were incubated at 4° C. for 30 min. After incubation, the cell mixture was washed 3 times, and then 200 μL of FACS buffer was added to each well to resuspend the cells. Detection was performed using a flow cytometer (Beckman, CytoFLEX AOO-1-1102).

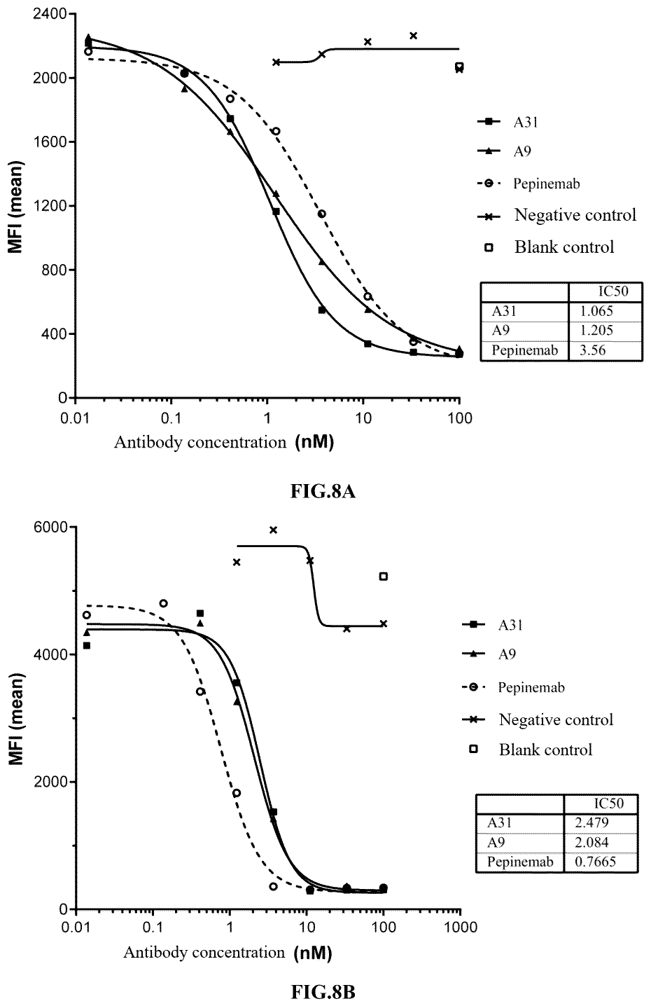

The results, as shown in FIGS. 8A-8B, indicated that the candidate antibodies effectively blocked the binding of HuCD100 antigen protein to HuPlexin-B1-HEK293 and HuPlexin-B2-HEK293 cells. Among them, the antibody molecules A9 and A31 exhibited superior blocking ability against the binding of HuCD100 antigen protein to HuPlexin-B1-HEK293 compared to the positive control antibody Pepinemab (FIG. 8A). The blocking ability of the antibody molecules A9 and A31 against the binding of HuCD100 antigen protein to HuPlexin-B2-HEK293 was weaker than that of the positive control antibody Pepinemab (FIG. 8B).

### Example 8 Humanization of Alpaca-Derived Antibodies

Compared to murine antibodies, alpaca-derived nanobodies share higher homology with human antibodies, but their structures are unique. Therefore, during the humanization design of A9 and A31, the AbM definition system was used to define CDR sequences and framework region sequences. The framework region sequences of each nanobodies that are closest to human germline genes were selected, and during the process of back mutations, the maintenance of the antibody structure was also considered. Ultimately, a series of humanized antibodies were designed. Humanized antibodies A9-VHH1, A9-VHH2, A9-VHH3, and A31-VHH1 were constructed, expressed, and purified using the procedures described in Example 4. The amino acid sequences of the humanized antibodies are shown in Table 4.

Humanized antibody proteins A9-VHH1, A9-VHH2, A9-VHH3, and A31-VHH1 were further identified by SDS-PAGE and SEC-HPLC (for specific procedures, see Examples 5.1 and 5.2).

The results, as shown in Table 5, indicated that all candidate antibodies tested by reduced gel had a purity of more than 95%. The monomeric purity of all candidate antibodies tested by SEC-HPLC (except for A9-VHH3 which was undetected) was more than 95%.

TABLE 4
Amino acid sequences of 4 humanized antibodies (SEQ ID NO:)

| Antibody Name | VHH | VHH-Fc |
|---|---|---|
| A9-VHH1 | 11 | 12 |
| A9-VHH2 | 13 | 14 |
| A9-VHH3 | 15 | 16 |
| A31-VHH1 | 17 | 18 |

TABLE 5
Expression levels and physicochemical
properties of the candidate antibodies

| | | | | Expression level | |
|---|---|---|---|---|---|
| No. | Clone | Species | SDS-PAGE | (μg/mL) | SEC |
| 1 | A9-VHH1 | alpaca | >95.0 | 78.80 | 98.51 |
| 2 | A9-VHH2 | alpaca | >95.0 | 69.10 | 95.55 |
| 3 | A9-VHH3 | alpaca | >95.0 | 65.30 | N/A |
| 4 | A31-VHH1 | alpaca | >95.0 | 50.10 | 98.33 |

N/A: Not detected.

### Example 9 Detection of Antigen-Binding Activity of Humanized Antibodies

In this example, the binding of humanized antibodies (A9-VHH1, A9-VHH2, A9-VHH3, and A31-VHH1) to CD100 antigen proteins HuCD100-His, MusCD100-His, and CynoCD100-His was detected by ELISA. Additionally, the binding ability of the expressed antibodies (A9-VHH1, A9-VHH2, A9-VHH3, and A31-VHH1) to CD100-overexpressing cells (HuCD100-HEK293, MusCD100-HEK293, CynoCD100-HEK293), HuPBMCs naturally expressing human CD100, and Jurkat cells naturally expressing human CD100 was evaluated by FACS.

### 9.1 ELISA-Based Detection of Antibody Binding to CD100 Antigen Proteins

In this example, the binding activity of humanized antibodies (A9-VHH1, A9-VHH2, A9-VHH3, and A31-VHH1) to CD100 antigen proteins HuCD100-His, MusCD100-His, and CynoCD100-His was evaluated. For specific detection procedures, see Example 6.1.

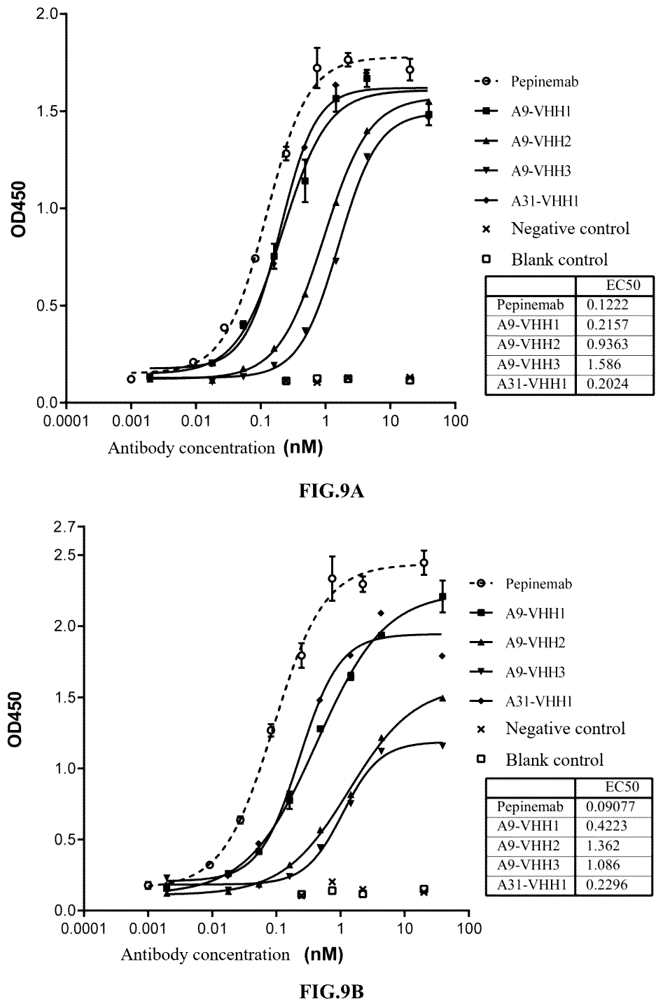

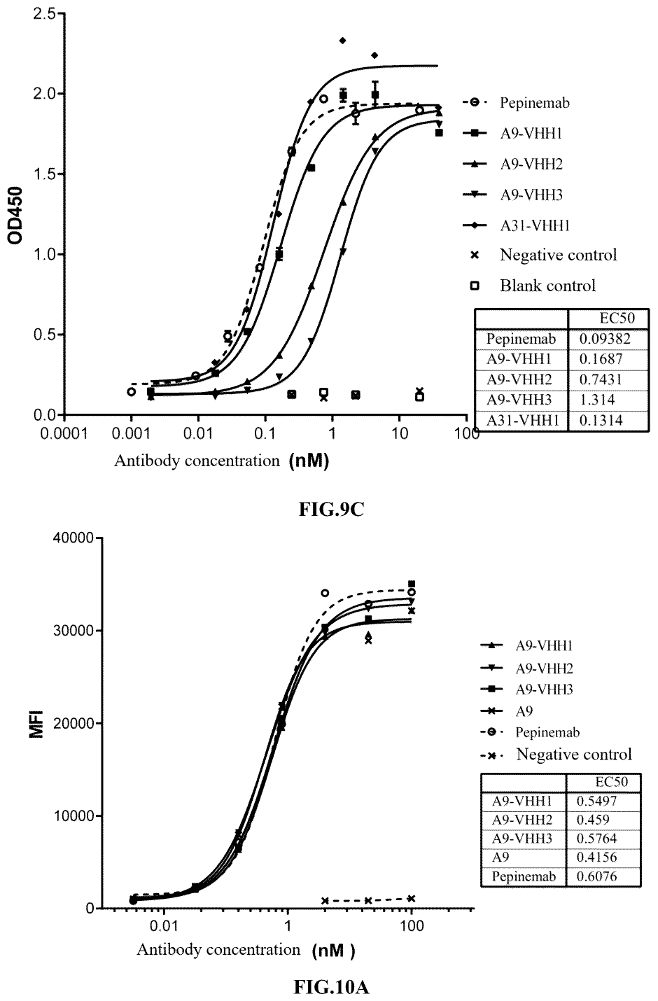

The results, as shown in FIGS. 9A-9C, indicated that all humanized antibody molecules exhibited weaker binding activity to HuCD100-His (FIG. 9A) and MusCD100-His (FIG. 9B) compared to the positive control antibody. Except for A31-VHH1, which exhibited binding activity to CynoCD100-His cells comparable to that of the positive control antibody, other humanized antibody molecules had weaker binding activity to CynoCD100-His compared to the positive control antibody (FIG. 9C).

### 9.2 FACS-Detection of Antibody Binding to CD100-HEK293 Cells

In this example, the binding activity of humanized antibodies (A9-VHH1, A9-VHH2, A9-VHH3, and A31-VHH1) was evaluated using human CD100-overexpressing cells (HuCD100-HEK293, MusCD100-HEK293, CynoCD100-HEK293). For specific detection procedures, see Example 6.2.

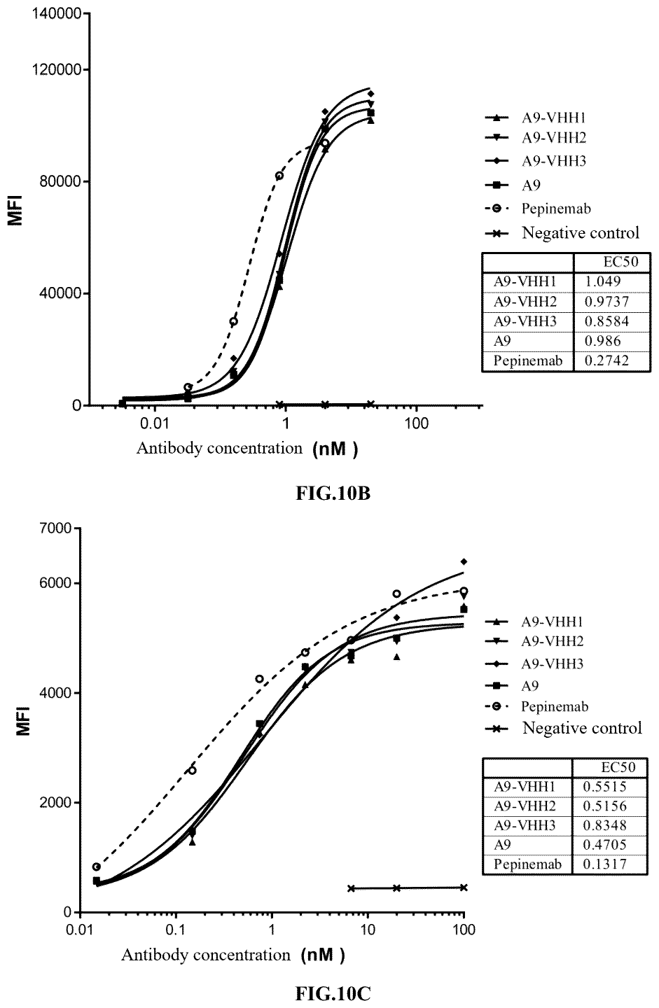

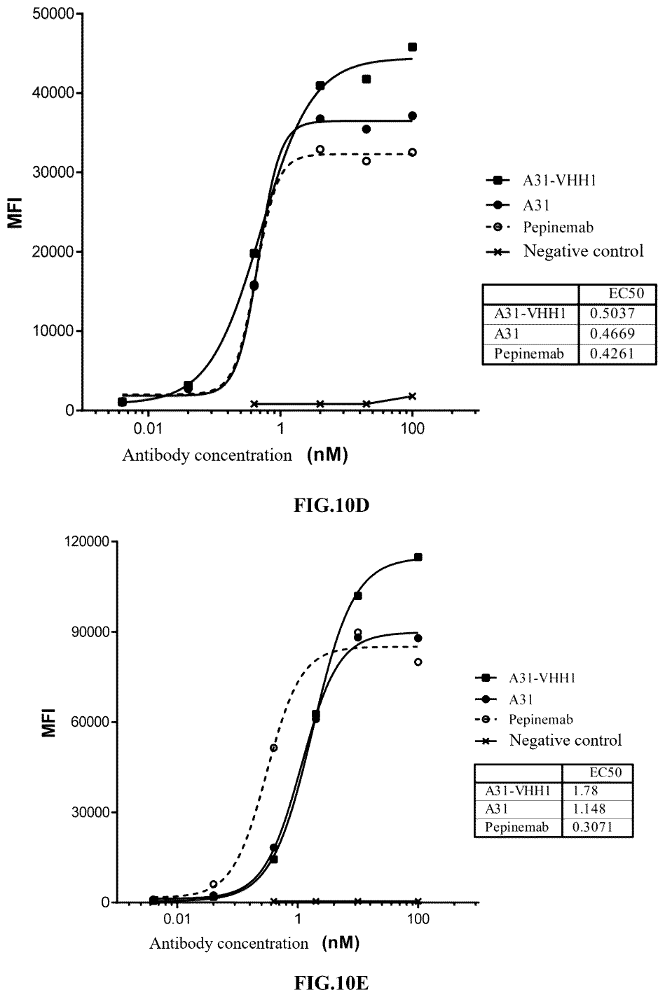

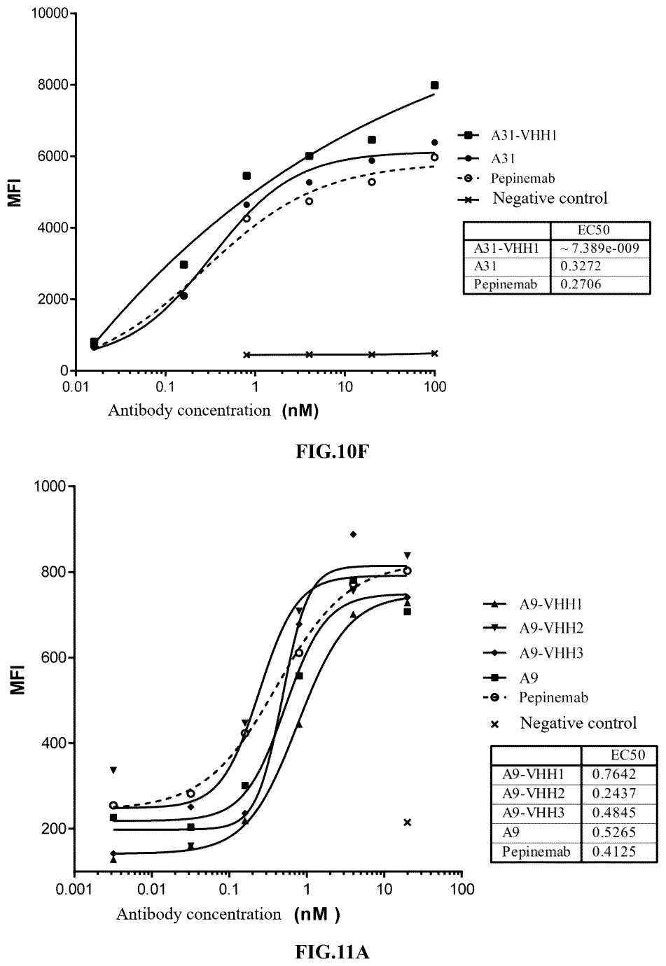

The results, as shown in FIGS. 10A-10F, indicated that the humanized A9 antibody molecules (A9-VHH1, A9-VHH2, A9-VHH3) exhibited binding activity to HuCD100-HEK293 cells comparable to that of the positive control antibody and parental antibody (FIG. 10A), and their binding activity to MusCD100-HEK293 (FIG. 10B) and CynoCD100-HEK293 (FIG. 10C) cells were also comparable to that of the parental antibody but weaker than that of the positive control antibody. The humanized A31 antibody molecule A31-VHH1 showed binding activity to HuCD100-HEK293 cells comparable to that of the positive control antibody and parental antibody (FIG. 10D), and its binding activity to MusCD100-HEK293 cells was comparable to that of the parental antibody but weaker than that of the positive control antibody (FIG. 10E). Moreover, A31-VHH1 exhibited some binding activity to CynoCD100-HEK293 cells (FIG. 10F).

### 9.3 FACS-Based Detection of Antibody Binding to HuPBMC and Jurkat Cells

In this example, the binding activity of humanized antibodies (A9-VHH1, A9-VHH2, A9-VHH3, and A31-VHH1) was evaluated using HuPBMCs and Jurkat cells. For specific procedures, see Example 6.3.

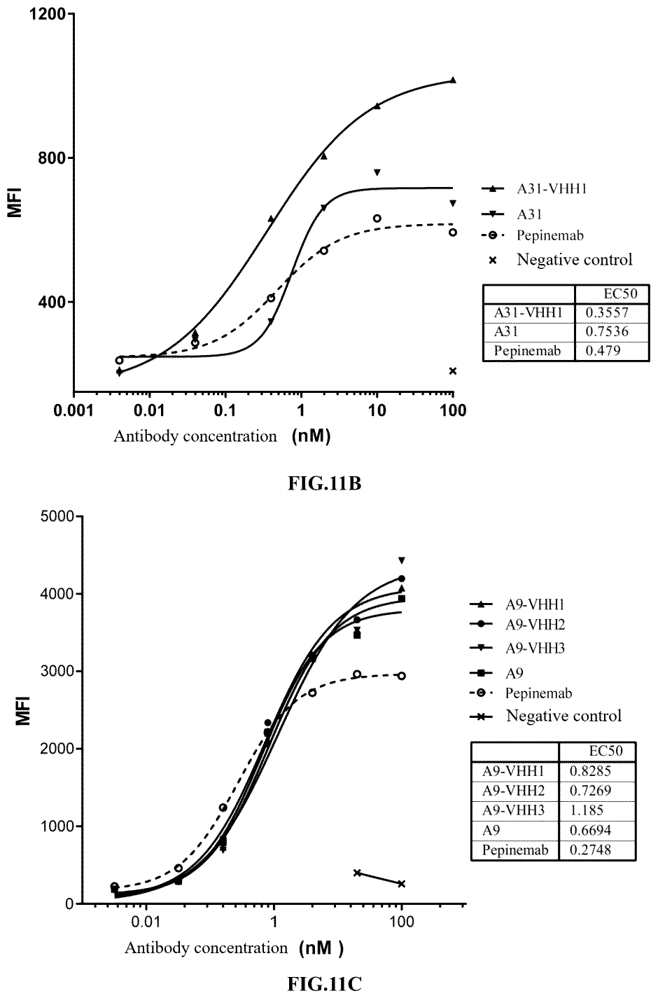

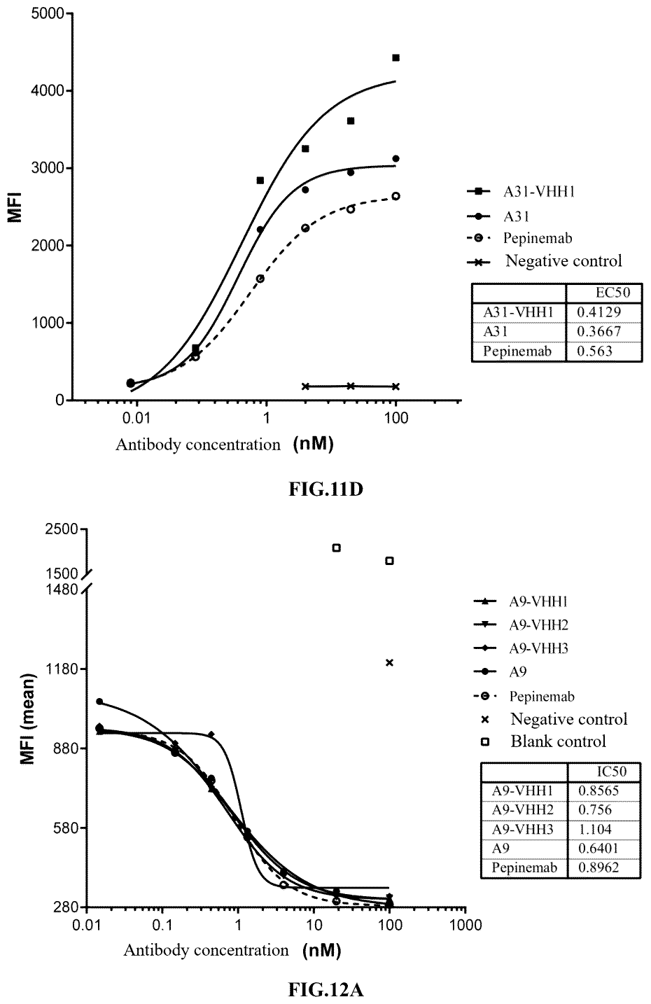

The results, as shown in FIGS. 11A-11D, indicated that humanized antibody molecules exhibited good binding activity to both HuPBMC and Jurkat cells. Among them, A31-VHH1 showed superior binding activity to HuPBMC (FIG. 11B) and Jurkat (FIG. 11D) cells compared to the positive control antibody. A9-VHH2 showed binding activity to HuPBMC cells comparable to that of the positive control antibody and parental antibody (FIG. 11A), but its binding activity to Jurkat cells was weaker than that of the positive control antibody (FIG. 11C). A9-VHH1 and A9-VHH3 showed weaker binding activity to both HuPBMC (FIG. 11A) and Jurkat (FIG. 11C) cells compared to the positive control antibody.

### Example 10 Blocking Activity Detection of Humanized Antibodies

In this example, the blocking activity of the candidate humanized antibodies (A9-VHH1, A9-VHH2, A9-VHH3, and A31-VHH1) was evaluated using three cell lines: HuPlexin-B1-HEK293, HuPlexin-B2-HEK293, and MusPlexin-B1-HEK293. For specific procedures, see Example 7.

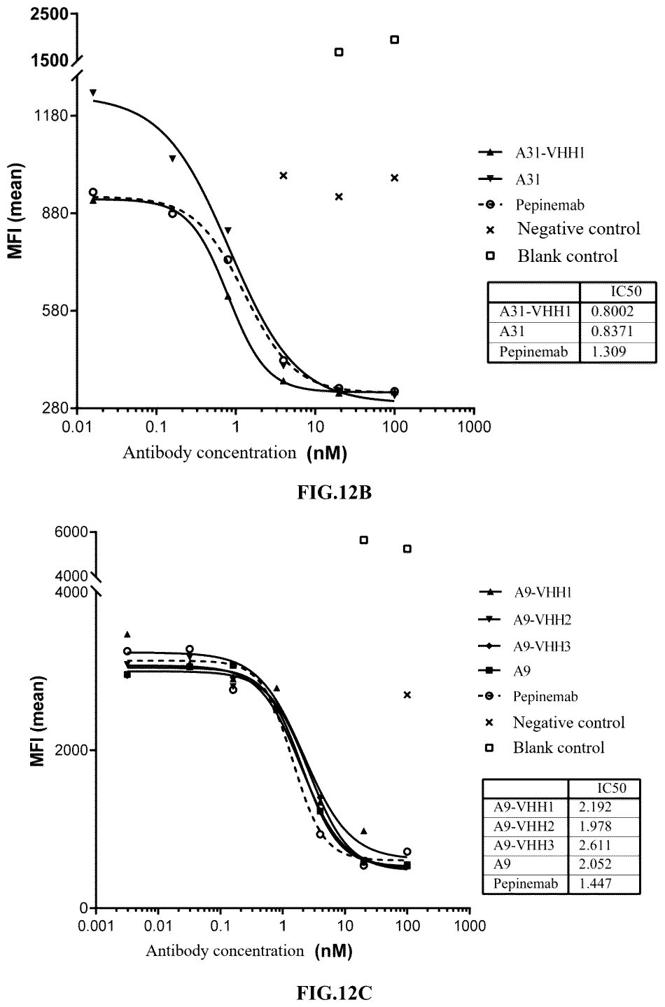

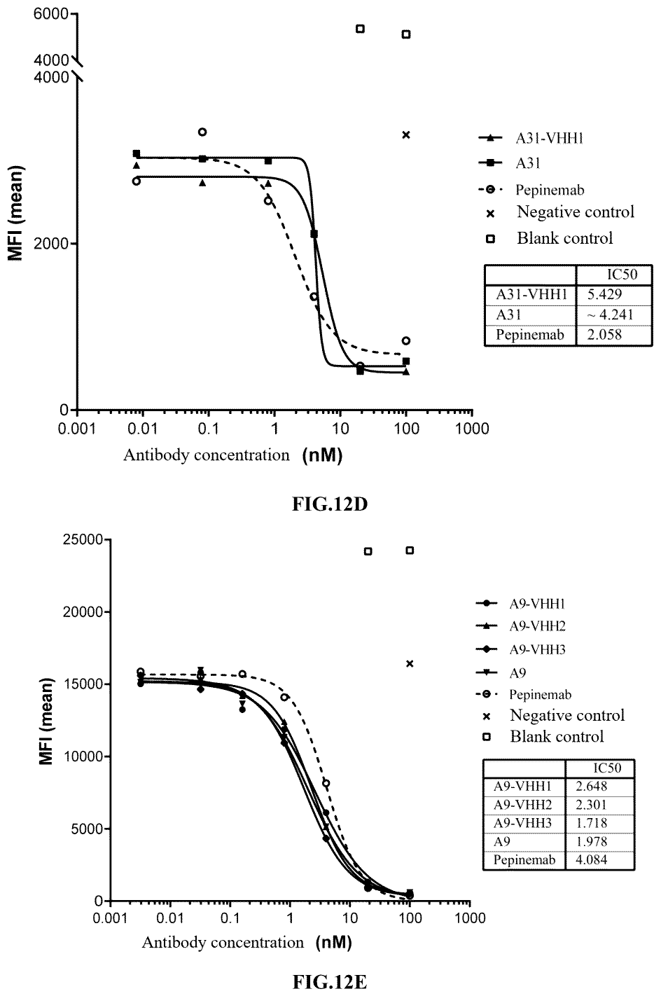

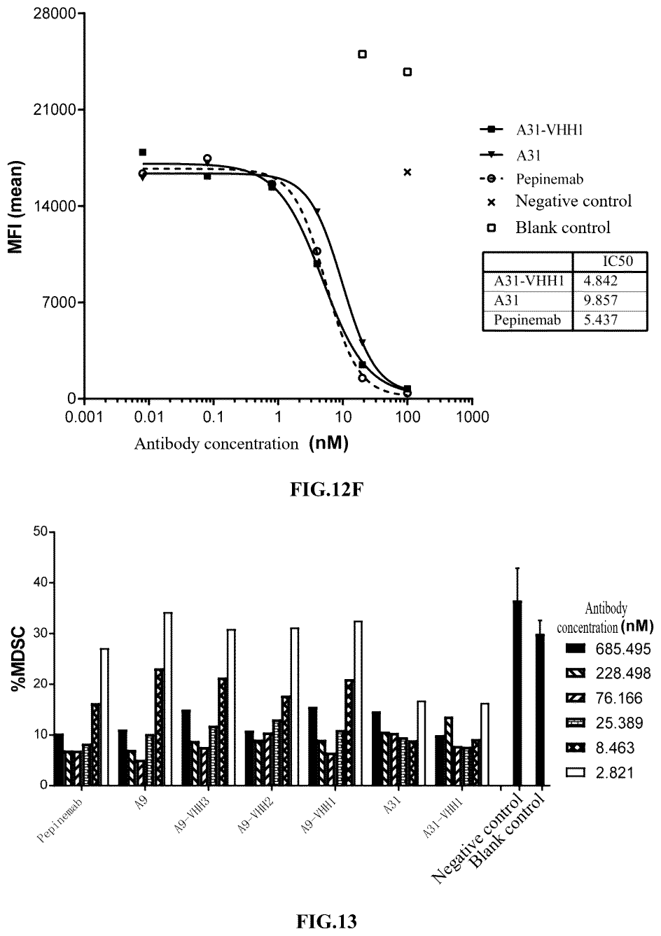

The results, as shown in FIGS. 12A-12F, indicated that humanized antibodies effectively blocked the binding of HuCD100-Fc antigen protein to HuPlexin-B1-HEK293 and HuPlexin-B2-HEK293 cells, as well as the binding of MusCD100-Fc antigen protein to MusPlexin-B1-HEK293 cells. Among them, A9-VHH1 and A9-VHH2 showed blocking ability against the binding of HuCD100-Fc to HuPlexin-B1-HEK293 cells comparable to that of the positive control antibody, whereas A9-VHH3 showed weaker blocking ability against the binding of HuCD100-Fc to HuPlexin-B1-HEK293 cells compared to the positive control antibody (FIG. 12A). A31-VHH1 exhibited superior blocking ability against the binding of HuCD100-Fc to HuPlexin-B1-HEK293 cells compared to the positive control antibody (FIG. 12B). All humanized molecules showed weaker blocking ability against the binding of HuCD100-Fc to HuPlexin-B2-HEK293 cells than the positive control antibody (FIGS. 12C-12D). All humanized antibodies showed superior blocking ability against the binding of MusCD100-Fc to MusPlexin-B1-HEK293 cells compared to the positive control antibody (FIGS. 12E-12F).

### Example 11 Detection of MDSC Proliferation Inhibition

This example evaluates the in vitro pharmacological efficacy of the candidate antibodies (A9-VHH1, A9-VHH2, A9-VHH3, and A31-VHH1) by examining their ability to inhibit the proliferation of myeloid-derived suppressor cells (MDSCs).

CD33+ cells were isolated from fresh PBMCs by using CD33 magnetic sorting beads (Miltenyi Biotech), and after sorting was completed, the cell density was adjusted to 1×10⁶/mL for later use. Antibodies were serially diluted with 1640 complete medium. The HuCD100-His antigen protein was diluted with 1640 complete medium and diluted to 400.0 g/mL. Antibody dilutions were mixed with HuCD100-His antigen protein dilutions at a 1:1 ratio, incubated at room temperature for 30 minutes. 96-well cell culture plates were used, 100 μL of CD33^{×} cells with a density of 1×10⁶/mL (1×10⁵ cells per well) were added to each well, and then 100 μL of antibody and the HuCD100-His antigen protein mixture were added to each well for a total of 200 μL. After incubating the cell culture plate in a 37° C. cell incubator for 72 h, the cells in sample well were transferred to a 96-well U-shaped plate, and the plate was washed twice with FACS buffer. Three direct-labeled antibodies PE-anti-human CD33 (Biolegend, 303404), FITC-anti-human HLA-DR (Biolegend, 307604), APC anti-human CD11b Antibody (Biolegend, 301310) were diluted 1:100 with FACS buffer, 100 μL of three direct-labeled antibody mixture were added to each well, and the plates were incubated at 4° C. for 30 min. The plates were washed twice with FACS buffer. Finally, detection was performed by a flow cytometer (Beckman, CytoFLEX AOO-1-1102).

The results, as shown in FIG. 13 and Table 6, indicated that the anti-CD100 antibodies A9, A9-VHH3, A9-VHH2, A9-VHH1, A31, A31-VHH1 and the control antibody Pepinemab can effectively neutralize the induction effect of CD100 on the MDSC populations. Among them, at low concentrations (8.463 nM and 2.821 nM), the antibody molecules A31 and A31-VHH1 exhibited superior inhibitory effects on MDSC proliferation compared to the positive control antibody, and the antibody molecules A9, A9-VHH1, A9-VHH2, and A9-VHH3 showed weaker inhibitory effects on MDSC proliferation than the positive control antibody; at high concentration (685.495 nM), the antibody molecules A9, A9-VHH2, and A31-VHH1 showed inhibitory effects on MDSC proliferation comparable to those of the positive control antibody.

TABLE 6
Inhibition of MDSC activity by anti-CD100 antibodies

| | MDSC proportion (%) at different antibody concentrations | | | | | |
|---|---|---|---|---|---|---|
| Group | 685.495 nM | 228.498 nM | 76.166 nM | 25.389 nM | 8.463 nM | 2.821 nM |
| Pepinemab | 10.30 | 6.97 | 6.92 | 8.29 | 16.30 | 27.14 |
| A9 | 11.11 | 7.08 | 5.12 | 10.24 | 23.16 | 34.25 |
| A9-VHH3 | 15.02 | 8.85 | 7.66 | 11.83 | 21.34 | 30.94 |
| A9-VHH2 | 10.90 | 9.09 | 10.52 | 13.08 | 17.81 | 31.22 |
| A9-VHH1 | 15.60 | 9.06 | 6.56 | 11.03 | 21.07 | 32.57 |
| A31 | 14.73 | 10.70 | 10.43 | 9.56 | 8.97 | 16.81 |
| A31-VHH1 | 10.00 | 13.65 | 7.84 | 7.71 | 9.21 | 16.35 |
| Negative | 41.01 | / | / | / | / | / |
| control | | | | | | |
| Blank | 28.17 | / | / | / | / | / |
| control | | | | | | |

### Example 12: Preparation of PD-L1 Antibody

In this example, the antibody NB22D-21-huVH2 (see CN112745391A) was subjected to affinity maturation modification to improve antibody affinity and other biological activities. The affinity maturation modification was based on the M13 phage display technology, using codon-based primers (during primer synthesis, individual codons were composed of NNK) to introduce mutations into the CDR regions, constructing four phage display libraries: Library 1: single-point combinatorial mutation (combinatorial mutation of CDR1+CDR2+CDR3); Libraries 2, 3, and 4: double-point combinatorial mutations (Library 2: double-point combinatorial mutations of CDR1+CDR3: Library 3: double-point combinatorial mutations of CDR2+CDR3; Library 4: double-point combinatorial mutations of CDR1+CDR2).

The specific library construction procedures were as follows: first, the primers containing point mutations were synthesized (Genewiz Biotechnology Co., Ltd.); second, the coding sequence of the antibody to be modified (NB22D-21-huVH2, hereinafter referred to as the parental antibody) served as the PCR amplification template to amplify the sequences of CDR regions containing mutations, and the fragments containing different CDR mutations were combined by bridge PCR; the point-mutated antibodies was then ligated into the phage display vector through double digestion (HindIII and NotI) and double sticky-end ligation; finally, the antibody sequences with mutation sites was transferred into Escherichia coli SS320 by electroporation. For the specific procedures for phage library preparation and library screening, see Example 3. The obtained antibody was named m18-VHH. The amino acid sequences of the variable regions of the resulting antibody are shown in Table 7, and the CDR sequences were determined using the AbM definition for CDRs.

TABLE 7
Amino acid sequences of anti-PD-L1 antibody
variable regions (SEQ ID NO:)

| Antibody name | CDR1 | CDR2 | CDR3 | VHH |
|---|---|---|---|---|
| m18-VHH | 20 | 21 | 22 | 23 |

### Example 13 Generation and Expression of Antibody m18

The fusion expression vector was constructed by linking the C-terminal of the VHH gene sequence to the N-terminal of the human IgGI Fc fragment gene sequence to fuse the m18-VHH antibody obtained by affinity maturation and the human IgGI Fc fragment, and the fusion expression vector plasmid was transformed into ExpiCHO cells to induce expression to obtain the VHH-Fc chimeric antibody protein fused with Fc fragment (SEQ ID NO: 24), hereinafter designated as m18 antibody.

The ExpiCHO transient expression system was used for the antibody expression, using ExpiCHO™ Expression Medium (Gibco, A29100-01) and Gibco™ ExpiFectamine™ CHO Transfection Kit (Gibco, A29129). For specific procedures, see Example 4.

### Example 14: Detection of Antigen-Binding Activity of Antibody m18

In this example, the binding ability of the affinity-matured VHH-Fc antibody to PD-L1-overexpressing cells (human PD-L1-CHO cells, human non-small cell lung cancer cell line HCC827 cells, mouse PD-L1-CHO cells, and cynomolgus monkey PD-L1-CHO cells) was detected by FACS. For the sources or preparation procedures of the above cell lines, see CN112745391A.

### 14.1 FACS-Based Detection of Antibody Binding Ability to Human PD-L1-CHO Cells

In this example, the human PD-L1-CHO cells were used to evaluate the binding activity of the antibody to human PD-L1-overexpressing cells.

The specific procedures were as follows: the cultured human PD-L1-CHO cells (for the preparation process of PD-L1-CHO cells, see Example 1 in CN112745391A) were harvested and centrifuged at 300 g to remove the supernatant. The cells were resuspended with the prepared FACS buffer and counted, and the cell suspension density was adjusted to 2×10⁶ cells/mL. 100 μL of human PD-L1-CHO cells was added per well to the 96-well plates. The plates were centrifuged at 300 g to remove the supernatant. The serially diluted antibody m18 and control antibody dilutions were added to the corresponding wells, and the cells were pipetted to mix with a multichannel pipette and incubated at 4° C. for 30 minutes. The incubated cell mixture was centrifuged at 300 g to remove the supernatant, 200 μL of FACS buffer was added to corresponding wells, and the cells were resuspended with a multichannel pipette. The centrifugation, resuspension and washing steps were repeated twice, and the cells were centrifuged at 300 g to remove the supernatant. The PE-labeled anti-human IgG Fc flow antibody (Abcam, ab98596) was added, and the cells were pipetted to mix with a multichannel pipette and incubated at 4° C. for 30 minutes. The cells were centrifuged at 300 g to remove the supernatant, and FACS buffer was added to resuspend the cells. The centrifugation, resuspension and washing steps were repeated twice, and 200 μL of FACS buffer was added per well to resuspend the cells. Detection was performed using a flow cytometer (Beckman, CytoFLEX AOO-1-1102). The positive control antibody used was Avelumab (preparation procedures as described in Patent WO2013079174).

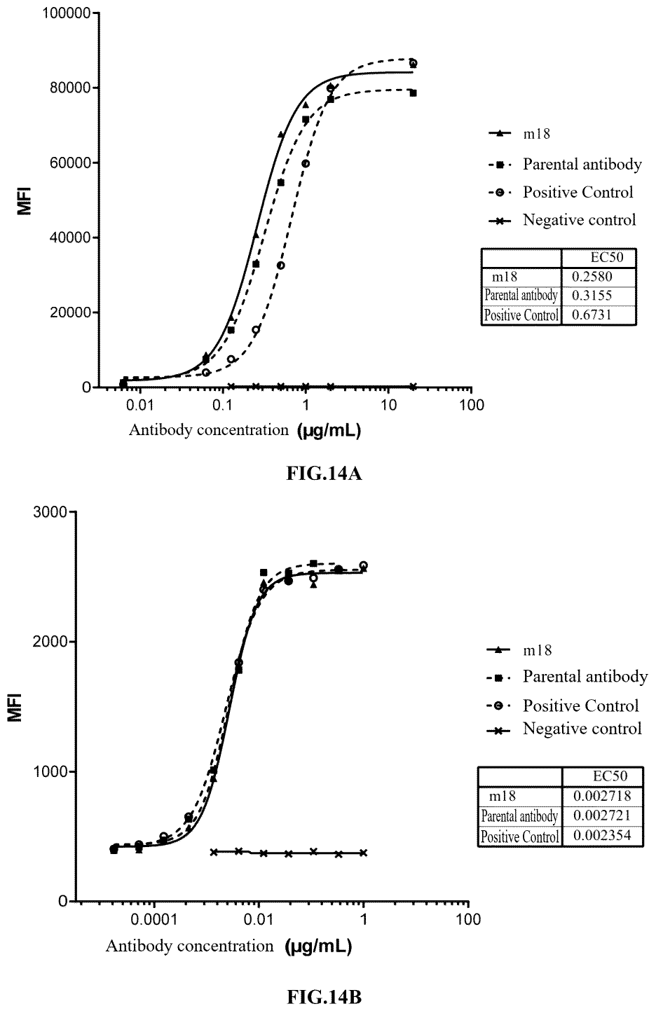

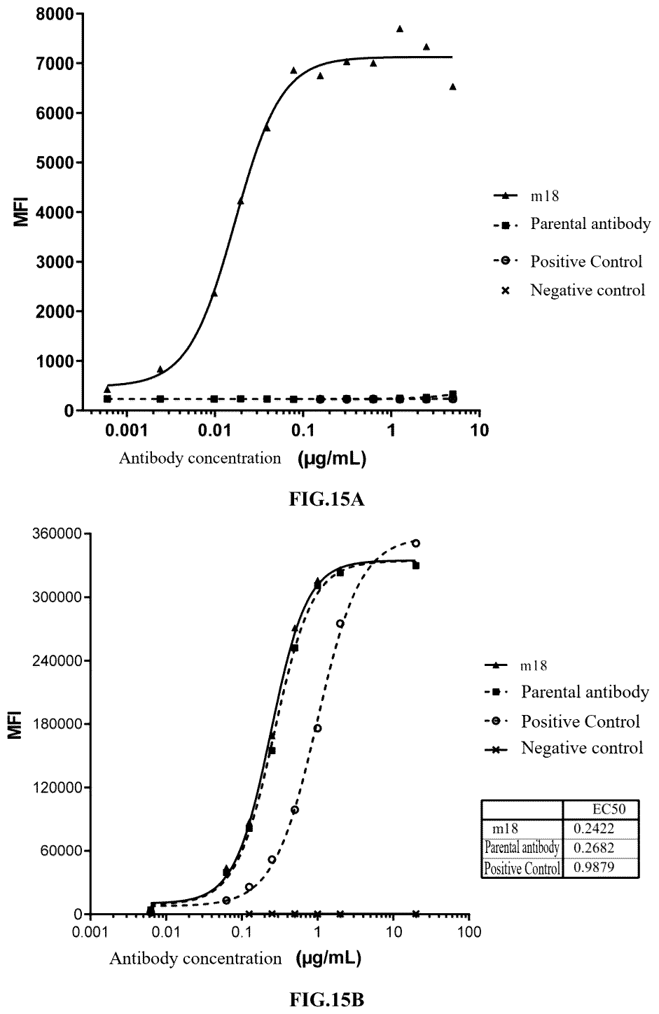

The results, as shown in FIG. 14A, indicated that the binding activity of antibody m18 to human PD-L1-CHO cells was superior to that of the parental antibody NB22D-21-huVH2 and the positive control antibody Avelumab.

### 14.2 FACS-Based Detection of Antibody Binding Ability to Human Non-Small Cell Lung Cancer Cell Line HCC827 Cells

In this example, human non-small cell lung cancer cell line HCC827 cells were used to evaluate the binding activity of the antibody to PD-L1 protein on human tumor cells.

The specific procedures were as follows: HCC827 cells (ATCC: CRL-2868) were digested with Trypsin containing 0.25% EDTA, harvested and centrifuged at 300 g to remove the supernatant. The cells were resuspended in the prepared FACS buffer and counted, and the cell suspension density was adjusted to 2×10⁶ cells/mL. 100 μL of HCC827 cells were added per well to 96-well plates. The plates were centrifuged at 300 g to remove the supernatant. The serially diluted antibody m18 and control antibody dilutions were added to the corresponding wells, and the cells were pipetted to mix with a multichannel pipette and incubated at 4° C. for 30 minutes. The incubated cell mixture was centrifuged at 300 g to remove the supernatant, 200 μL of FACS buffer was added to corresponding wells, and the cells were resuspended with a multichannel pipette. The centrifugation, resuspension and washing steps were repeated twice, and the cells were centrifuged at 300 g to remove the supernatant. 100 μL of m18 antibody and control antibody dilutions (1 μg/mL) were added to the corresponding wells, the cells were resuspended, and incubated at 4° C. for 30 minutes. The incubated cell mixture was centrifuged at 300 g to remove the supernatant, 200 μL of FACS buffer was added to corresponding wells, and the cells were resuspended with a multichannel pipette. The centrifugation, resuspension and washing steps were repeated twice, and the cells were centrifuged at 300 g to remove the supernatant. The PE-labeled anti-biotin flow antibody (Abcam) was added, and the cells were pipetted to mix with a multichannel pipette and incubated at 4° C. for 30 minutes. The cells were centrifuged at 300 g to remove the supernatant, and FACS buffer was added to resuspend the cells. The centrifugation, resuspension and washing steps were repeated twice, and 200 μL of FACS buffer was added per well to resuspend the cells. Detection was performed using a flow cytometer (Beckman, CytoFLEX AOO-1-1102).

The results, as shown in FIG. 14B, indicated that the binding activity of antibody m18 to human non-small cell lung cancer cell line HCC827 cells was comparable to that of the parental antibody NB22D-21-huVH2 and the positive control antibody Avelumab.

### 14.3 FACS-Based Detection of Antibody Binding Ability to Mouse PD-L1-CHO Cells and Cynomolgus Monkey PD-L1-CHO Cells

In this example, mouse PD-L1-CHO cells and cynomolgus monkey PD-L1-CHO cells were used to evaluate cross-binding activity with monkey and mouse PD-L1.

The specific procedures were as follows: the cultured mouse PD-L1-CHO cells and cynomolgus monkey PD-L1-CHO cells (for the preparation process of mouse PD-L1-CHO cells and cynomolgus monkey PD-L1-CHO cells, see Example 1 of CN112745391A) were harvested and centrifuged at 300 g to remove the supernatant. The cells were resuspended with the prepared FACS buffer and counted, and the cell suspension density was adjusted to 2×10⁶ cells/mL. 100 μL of mouse PD-L1-CHO cells and cynomolgus monkey PD-L1-CHO cells were added per well to the 96-well plates, respectively. The plates were centrifuged at 300 g to remove the supernatant. The serially diluted antibody m18 and control antibody dilutions were added to the corresponding wells, and the cells were pipetted to mix with a multichannel pipette and incubated at 4° C. for 30 minutes. The incubated cell mixture was centrifuged at 300 g to remove the supernatant, 200 μL of FACS buffer was added to corresponding wells, and the cells were resuspended with a multichannel pipette. The centrifugation, resuspension and washing steps were repeated twice, and the cells were centrifuged at 300 g to remove the supernatant. The PE-labeled anti-human IgG Fc flow antibody (Abcam, ab98596) was added, and the cells were pipetted to mix with a multichannel pipette and incubated at 4° C. for 30 minutes. The cells were centrifuged at 300 g to remove the supernatant, and FACS buffer was added to resuspend the cells. The centrifugation, resuspension and washing steps were repeated twice, and 200 μL of FACS buffer was added per well to resuspend the cells. Detection was performed using a flow cytometer (Beckman, CytoFLEX AOO-1-1102).

The results, as shown in FIGS. 15A-15B, indicated that the m18 antibody exhibited good binding activity to mouse PD-L1-CHO cells, while the parental antibody and positive control antibody did not bind to mouse PD-L1-CHO cells (FIG. 15A), suggesting that the m18 molecule could be used in animal model experiments with Balb/C mice; the binding activity of the m18 antibody to cynomolgus monkey PD-L1-CHO cells was superior to that of the positive control antibody and comparable to that of the parental antibody (FIG. 15B).

### Example 15 Specificity Detection of Antibody m18 Binding to PD-L1

In this example, the ELISA method was used to detect the binding activity of the affinity-matured VHH-Fc antibody to other proteins in the B7 family to evaluate the specificity of antibody m18 for the PD-L1 protein. The specific procedures were as follows:

One day before the experiment, 30 μL of protein dilutions with a final concentration of 2 μg/mL (B7-H1 (i.e., PD-L1), B7-H2, B7-H3, B7-H4, and B7-DC (all proteins were purchased from Sino Biological Inc., catalog numbers 10084-HNAH, 11559-H08H, 11188-H08H, 10738-H08H, and 10292-H08H-B, respectively)) were added to the ELISA plates and the plates were incubated overnight at 4° C. The next day, the ELISA plate was rinsed three times with PBST, then blocked with 150 μL of PBS containing 5% skim milk powder at room temperature for 2 hours. After washing the plates three times with PBST, 30 μL of m18 antibody dilution and control antibody dilution were added to the ELISA plates and the plates were incubated at room temperature for 1 hour. The plates were washed three times with PBST, and 30 μL of 1:7000 diluted anti-human IgG Fc-HRP secondary antibody was added per well, followed by incubation at room temperature for 30 minutes. After washing the plate six times with PBST, TMB was added for color development, and the reaction was terminated by adding 2M HCl. The absorbance was read at a wavelength of 450 nM using a microplate reader (Molecular Devices, SpecterMax 190).

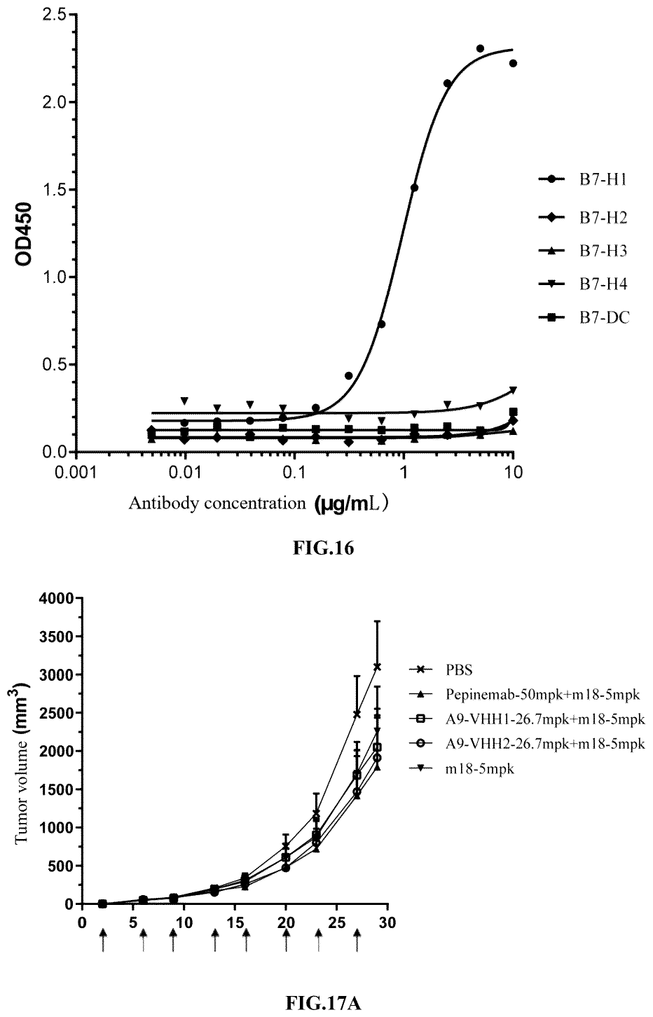

The results, as shown in Table 8 and FIG. 16, indicated that after affinity maturation, the antibody m18 did not exhibit binding activity to B7 family molecules other than B7-H1 (i.e., PD-L1) and only bound to B7-H1. This binding specificity was consistent with that of the parental antibody.

TABLE 8
Specificity of the candidate molecule
for binding to the PD-L1 protein

Protein

| Clone | B7-H1 | B7-H2 | B7-H3 | B7-H4 | B7-DC |
|---|---|---|---|---|---|
| m18 | + | N/A | N/A | N/A | N/A |

+: Binding activity detected. N/A: No binding activity detected.

### Example 16 Efficacy Evaluation in CT-26 Animal Model

This example detected the in vivo anti-tumor effect of two candidate humanized anti-CD100 antibodies (A9-VHH1 and A9-VHH2) in combination with the anti-PD-L1 antibody. The tumor cells used were CT-26 (Shanghai Cell Bank of Chinese Academy of Sciences, TCM37), and Pepinemab was used as the positive control.

The specific procedures were as follows: Female Balb/C mice aged 6-8 weeks and weighing about 20 g (Beijing Vital River Laboratory Animal Technology Co., Ltd.) were subcutaneously injected with 5x105 CT-26 cells unilaterally. Two days after tumor inoculation, the mice were randomly grouped and caged separately. There were 8 tumor-bearing nude mice in each group, a total of 5 groups, including the PBS negative control group, the candidate antibody combination groups (antibody A9-VHH1+antibody m18, antibody A9-VHH2+antibody m18), and the positive or reference control antibody groups (antibody Pepinemab+antibody m18, antibody m18). The administration dose of the anti-CD100 antibodies was 26.7 mpk, and the administration dose of the anti-PD-L1 antibody was 5 mpk. All administrations were performed by intraperitoneal injection twice weekly, and the tumor volume was measured twice weekly, for a total of 8 doses over 4 weeks (BIW*4). The tumor volume (V) was calculated as: V=L×W²/2 (where L is the longest tumor diameter and W is the shortest tumor diameter). The tumor growth inhibition rate (TGI) was calculated as: TGI=1−T/C (%). T/C % is the relative tumor proliferation rate, that is, the percentage ratio of the relative tumor volume between the treatment group and the control group at a certain time point. One week after the end of administration, the mice were euthanized, and tumor tissues were collected. The data on tumor volume and mouse body weight changes were analyzed, and the tumor growth inhibition rate was calculated.

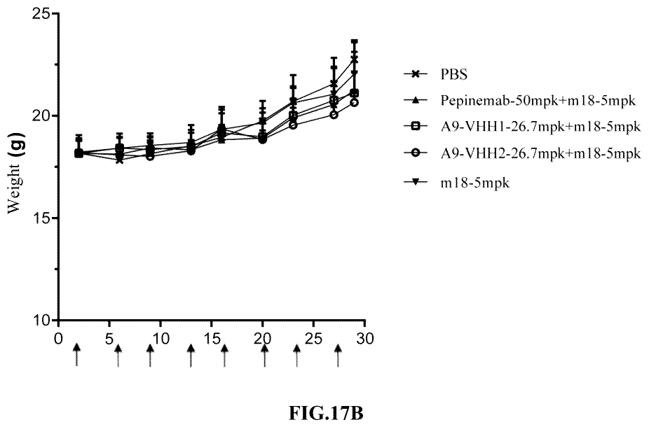

The results, as shown in Table 9, FIGS. 17A-17B, indicated that anti-CD100 antibodies A9-VHH2 and A9-VHH1 in combination with anti-PD-L1 antibody m18 further synergistically inhibited or delayed tumor growth compared to treatment with the anti-PD-L1 antibody m18 alone. This demonstrated that A9-VHH2 and A9-VHH1 antibodies in combination with anti-PD-L1 antibody m18 significantly improved the response rate to m18 antibody monotherapy in the mice inoculated with CT-26 and prolonged the survival, suggesting that such a combination could enhance the therapeutic effect of PD-L1 tumor immunotherapy. Meanwhile, after administration, all mice showed weight gain, and there was no significant difference in body weight among the experimental groups and the control group. This indicated that the antibodies did not cause obvious toxic and side effects on the mice and were safe.

TABLE 9
In vivo efficacy results of tumor growth inhibition rate (TGI, %)

| | Days | | | | | | | |
|---|---|---|---|---|---|---|---|---|
| | 6 | 9 | 13 | 16 | 20 | 23 | 27 | 29 |
| PBS | 0.00 | 0.00 | 0.00 | 0.00 | 0.00 | 0.00 | 0.00 | 0.00 |
| Pepinemab + m18 | 0.00 | 0.11 | 5.20 | 30.65 | 39.30 | 41.65 | 44.49 | 45.25 |
| A9-VHH1 + m18 | 0.00 | 11.83 | 2.58 | 10.57 | 21.28 | 24.76 | 32.55 | 35.50 |
| A9-VHH2 + m18 | 0.00 | 22.02 | 37.48 | 36.82 | 47.33 | 42.97 | 49.47 | 47.41 |
| m18 | 0.00 | 10.15 | 4.96 | 15.68 | 22.83 | 28.29 | 31.84 | 28.25 |

Those skilled in the art will appreciate that numerous modifications and variations of the present invention can be made without departing from its spirit and scope. The specific embodiments described herein are provided by way of example only and are not intended to limit the invention in any way. The real scope and spirit of the invention are indicated by the appended claims, and the specification and examples are merely illustrative.

Sequence Listing
A9-VHH

| Sequence ID | Name | Sequence |
|---|---|---|
| SEQ ID NO: 1 | CDR1 | GFSLDYYAIG |
| SEQ ID NO: 2 | CDR2 | CISSSGGSTD |
| SEQ ID NO: 3 | CDR3 | GVSGSVCPPLIGAY |
| SEQ ID NO: 4 | VHH | QVQLVESGGGLVQAGGSLRLSCAASGFSLDYYAIGWFRQAPGK |
| | | EREEVSCISSSGGSTDYADSVKGRFTISRAYAKVYLQMNSLKPED |
| | | TAVYYCAAGVSGSVCPPLIGAYWGQGTQVTVSS |

A31-VHH

| Sequence ID | Name | Sequence |
|---|---|---|
| SEQ ID NO: 5 | CDR1 | GFPLDYYAIG |
| SEQ ID NO: 6 | CDR2 | CIISGGSTK |
| SEQ ID NO: 7 | CDR3 | SWSCDLFPRDTYGWDA |
| SEQ ID NO: 8 | VHH | EVQLVESGGGLVQPEGSLRLSCAASGFPLDYYAIGWFRQAPGKE |
| | | REGVSCIISGGSTKYSASVKGRFTISRDSANNTVYLQMNSLKPED |
| | | TAVYFCAVSWSCDLFPRDTYGWDAWGQGTLVTVSS |

A9

| Sequence ID | Name | Sequence |
|---|---|---|
| SEQ ID NO: 9 | VHH-Fc | QVQLVESGGGLVQAGGSLRLSCAASGFSLDYYAIGWFRQAPGK |
| | | EREEVSCISSSGGSTDYADSVKGRFTISRAYAKVYLQMNSLKPED |
| | | TAVYYCAAGVSGSVCPPLIGAYWGQGTQVTVSSESKYGPPCPPC |
| | | PAPEFLGGPSVFLFPPKPKDTLMISRTPEVTCVVVDVSQEDPEVQ |
| | | FNWYVDGVEVHNAKTKPREEQFNSTYRVVSVLTVLHQDWLNG |
| | | KEYKCKVSNKGLPSSIEKTISKAKGQPREPQVYTLPPSQEEMTKN |
| | | QVSLTCLVKGFYPSDIAVEWESNGQPENNYKTTPPVLDSDGSFFL |
| | | YSRLTVDKSRWQEGNVFSCSVMHEALHNHYTQKSLSLSLGK |

A31

| Sequence ID | Name | Sequence |
|---|---|---|
| SEQ ID NO: 10 | VHH-Fc | EVQLVESGGGLVQPEGSLRLSCAASGFPLDYYAIGWFRQAPGKE |
| | | REGVSCIISGGSTKYSASVKGRFTISRDSANNTVYLQMNSLKPED |
| | | TAVYFCAVSWSCDLFPRDTYGWDAWGQGTLVTVSSESKYGPPC |
| | | PPCPAPEFLGGPSVFLFPPKPKDTLMISRTPEVTCVVVDVSQEDPE |
| | | VQFNWYVDGVEVHNAKTKPREEQFNSTYRVVSVLTVLHQDWL |
| | | NGKEYKCKVSNKGLPSSIEKTISKAKGQPREPQVYTLPPSQEEMT |
| | | KNQVSLTCLVKGFYPSDIAVEWESNGQPENNYKTTPPVLDSDGS |
| | | FFLYSRLTVDKSRWQEGNVFSCSVMHEALHNHYTQKSLSLSLG |
| | | K |

A9-VHH1

| Sequence ID | Name | Sequence |
|---|---|---|
| SEQ ID NO: 11 | VHH | QVQLVESGGGLVQPGGSLRLSCAASGFSLDYYAIGWFRQAPGKE |
| | | REEVSCISSSGGSTDYADSVKGRFTISRAYAKVYLQMNSLKPEDT |
| | | AVYYCAAGVSGSVCPPLIGAYWGQGTLVTVSS |
| SEQ ID NO: 12 | VHH-Fc | QVQLVESGGGLVQPGGSLRLSCAASGFSLDYYAIGWFRQAPGKE |
| | | REEVSCISSSGGSTDYADSVKGRFTISRAYAKVYLQMNSLKPEDT |
| | | AVYYCAAGVSGSVCPPLIGAYWGQGTLVTVSSESKYGPPCPPCP |
| | | APEFLGGPSVFLFPPKPKDTLMISRTPEVTCVVVDVSQEDPEVQF |
| | | NWYVDGVEVHNAKTKPREEQFNSTYRVVSVLTVLHQDWLNGK |
| | | EYKCKVSNKGLPSSIEKTISKAKGQPREPQVYTLPPSQEEMTKNQ |
| | | VSLTCLVKGFYPSDIAVEWESNGQPENNYKTTPPVLDSDGSFFLY |
| | | SRLTVDKSRWQEGNVFSCSVMHEALHNHYTQKSLSLSLGK |

A9-VHH

| Sequence ID | Name | Sequence |
|---|---|---|
| SEQ ID NO: 13 | VHH | QVQLVESGGGLVQAGGSLRLSCAASGFSLDYYAIGWFRQAPGK |
| | | EREEVSCISSSGGSTDYADSVKGRFTISRAYAKVYLQMNSLRAE |
| | | DTAVYYCAAGVSGSVCPPLIGAYWGQGTLVTVSS |
| SEQ ID NO: 14 | VHH-Fc | QVQLVESGGGLVQAGGSLRLSCAASGFSLDYYAIGWFRQAPGK |
| | | EREEVSCISSSGGSTDYADSVKGRFTISRAYAKVYLQMNSLRAE |
| | | DTAVYYCAAGVSGSVCPPLIGAYWGQGTLVTVSSESKYGPPCPP |
| | | CPAPEFLGGPSVFLFPPKPKDTLMISRTPEVTCVVVDVSQEDPEV |
| | | QFNWYVDGVEVHNAKTKPREEQFNSTYRVVSVLTVLHQDWLN |
| | | GKEYKCKVSNKGLPSSIEKTISKAKGQPREPQVYTLPPSQEEMTK |
| | | NQVSLTCLVKGFYPSDIAVEWESNGQPENNYKTTPPVLDSDGSF |
| | | FLYSRLTVDKSRWQEGNVFSCSVMHEALHNHYTQKSLSLSLGK |
| SEQ ID NO: 15 | VHH | QVQLVESGGGLVQAGGSLRLSCAASGFSLDYYAIGWFRQAPGK |
| | | GLEEVSCISSSGGSTDYADSVKGRFTISRAYAKVYLQMNSLRAE |
| | | DTAVYYCAAGVSGSVCPPLIGAYWGQGTLVTVSS |
| SEQ ID NO: 16 | VHH-Fc | QVQLVESGGGLVQAGGSLRLSCAASGFSLDYYAIGWFRQAPGK |
| | | GLEEVSCISSSGGSTDYADSVKGRFTISRAYAKVYLQMNSLRAE |
| | | DTAVYYCAAGVSGSVCPPLIGAYWGQGTLVTVSSESKYGPPCPP |
| | | CPAPEFLGGPSVFLFPPKPKDTLMISRTPEVTCVVVDVSQEDPEV |
| | | QFNWYVDGVEVHNAKTKPREEQFNSTYRVVSVLTVLHQDWLN |
| | | GKEYKCKVSNKGLPSSIEKTISKAKGQPREPQVYTLPPSQEEMTK |
| | | NQVSLTCLVKGFYPSDIAVEWESNGQPENNYKTTPPVLDSDGSF |
| | | FLYSRLTVDKSRWQEGNVFSCSVMHEALHNHYTQKSLSLSLGK |

A31-VHH1

| Sequence ID | Name | Sequence |
|---|---|---|
| SEQ ID NO: 17 | VHH | EVQLVESGGGLVQPEGSLRLSCAASGFPLDYYAIGWFRQAPGKE |
| | | REGVSCIISGGSTKYSASVKGRFTISRDSAKNTVYLQMNSLRAED |
| | | TAVYFCAVSWSCDLFPRDTYGWDAWGQGTLVTVSS |
| SEQ ID NO: 18 | VHH-Fc | EVQLVESGGGLVQPEGSLRLSCAASGFPLDYYAIGWFRQAPGKE |
| | | REGVSCIISGGSTKYSASVKGRFTISRDSAKNTVYLQMNSLRAED |
| | | TAVYFCAVSWSCDLFPRDTYGWDAWGQGTLVTVSSESKYGPPC |
| | | PPCPAPEFLGGPSVFLFPPKPKDTLMISRTPEVTCVVVDVSQEDPE |
| | | VQFNWYVDGVEVHNAKTKPREEQFNSTYRVVSVLTVLHQDWL |
| | | NGKEYKCKVSNKGLPSSIEKTISKAKGQPREPQVYTLPPSQEEMT |
| | | KNQVSLTCLVKGFYPSDIAVEWESNGQPENNYKTTPPVLDSDGS |
| | | FFLYSRLTVDKSRWQEGNVFSCSVMHEALHNHYTQKSLSLSLG |
| | | K |
| SEQ ID NO: 19 | IgG4SP Fc | ESKYGPPCPPCPAPEFLGGPSVFLFPPKPKDTLMISRTPEVTCV |
| | | VVDVSQEDPEVQFNWYVDGVEVHNAKTKPREEQFNSTYRV |
| | | VSVLTVLHQDWLNGKEYKCKVSNKGLPSSIEKTISKAKGQPR |
| | | EPQVYTLPPSQEEMTKNQVSLTCLVKGFYPSDIAVEWESNGQ |
| | | PENNYKTTPPVLDSDGSFFLYSRLTVDKSRWQEGNVFSCSVM |
| | | HEALHNHYTQKSLSLSLGK |

m18

| Sequence ID | Name | Sequence |
|---|---|---|
| SEQ ID NO: 20 | CDR1 | RTDSNIHGMH |
| SEQ ID NO: 21 | CDR2 | MIFIDGNTI |
| SEQ ID NO: 22 | CDR3 | DSRGYGRA |
| SEQ ID NO: 23 | VHH | EVQLVESGGGLVQPGGSLRLSCAASRTDSNIHGMHWYRQAP |
| | | GKGREWVGMIFIDGNTIVTDSVKGRFTISRDNAKNTLYLQMN |
| | | TLRAEDTAVYYCAADSRGYGRAWGQGTTVTVSS |
| SEQ ID NO: 24 | VHH-Fc | EVQLVESGGGLVQPGGSLRLSCAASRTDSNIHGMHWYRQAP |
| | | GKGREWVGMIFIDGNTIVTDSVKGRFTISRDNAKNTLYLQMN |
| | | TLRAEDTAVYYCAADSRGYGRAWGQGTTVTVSSEPKSCDKT |
| | | HTCPPCPAPELLGGPSVFLFPPKPKDTLMISRTPEVTCVVVDV |
| | | SHEDPEVKFNWYVDGVEVHNAKTKPREEQYNSTYRVVSVLT |
| | | VLHQDWLNGKEYKCKVSNKALPAPIEKTISKAKGQPREPQV |
| | | YTLPPSRDELTKNQVSLTCLVKGFYPSDIAVEWESNGQPENN |
| | | YKTTPPVLDSDGSFFLYSKLTVDKSRWQQGNVFSCSVMHEA |
| | | LHNHYTQKSLSLSPGK |

1. An antibody or an antigen-binding fragment thereof against CD100, comprising an immunoglobulin single variable domain, wherein
the single variable domain comprises three CDR sequences in the amino acid sequence of the single variable domain as set forth in SEQ ID NO: 4 or a variant thereof; or
the single variable domain comprises three CDR sequences in the amino acid sequence of the single variable domain as set forth in SEQ ID NO: 8 or a variant thereof;
wherein the variant differs from the CDR sequence from which it is derived by addition, deletion or substitution of no more than 2 amino acids.

2. The antibody or antigen-binding fragment thereof according to claim 1, wherein the single variable domain comprises:
(a) a CDR1 sequence as set forth in SEQ ID NO: 1; a CDR2 sequence as set forth in SEQ ID NO: 2; and a CDR3 sequence as set forth in SEQ ID NO: 3; or
(b) a CDR1 sequence as set forth in SEQ ID NO: 5; a CDR2 sequence as set forth in SEQ ID NO: 6; and a CDR3 sequence as set forth in SEQ ID NO: 7.

3. The antibody or antigen-binding fragment thereof according to claim 1, wherein the immunoglobulin single variable domain is a variable domain of a heavy chain antibody (VHH).

4. The antibody or antigen-binding fragment thereof according to claim 1, wherein the antibody or antigen-binding fragment thereof comprises an immunoglobulin single variable domain, wherein
the single variable domain comprises an amino acid sequence as set forth in SEQ ID NO: 4 or an amino acid sequence having at least 85%, at least 90%, at least 95% or higher sequence identity to SEQ ID NO: 4; or
the single variable domain comprises an amino acid sequence as set forth in SEQ ID NO: 8 or an amino acid sequence having at least 85%, at least 90%, at least 95% or higher sequence identity to SEQ ID NO: 8.

5. The antibody or antigen-binding fragment thereof according to claim 1, which is a single domain antibody, a heavy chain antibody, a humanized antibody or a chimeric antibody.

6. The antibody or antigen-binding fragment thereof according to claim 1, wherein the immunoglobulin single variable domain is capable of being fused to an additional molecule;
preferably, the additional molecule is an Fc domain of an immunoglobulin;
more preferably, the additional molecule is an Fc domain of an immunoglobulin G4 (IgG4SP).

7. The antibody or antigen-binding fragment thereof according to claim 6, wherein
the antibody or antigen-binding fragment thereof comprises an amino acid sequence as set forth in SEQ ID NO: 9 or an amino acid sequence having at least 85%, at least 90%, at least 95% or higher sequence identity to SEQ ID NO: 9; or
the antibody or antigen-binding fragment thereof comprises an amino acid sequence as set forth in SEQ ID NO: 10 or an amino acid sequence having at least 85%, at least 90%, at least 95% or higher sequence identity to SEQ ID NO: 10.

8. The antibody or antigen-binding fragment thereof according to claim 1, which is a humanized antibody or an antigen-binding fragment thereof.

9. The antibody or antigen-binding fragment thereof according to claim 8, wherein the humanized antibody or antigen-binding fragment thereof comprises an immunoglobulin single variable domain, wherein
the single variable domain comprises an amino acid sequence as set forth in SEQ ID NO: 11, 13 or 15 or an amino acid sequence having at least 85%, at least 90%, at least 95% or higher sequence identity to SEQ ID NO: 11, 13 or 15; or
the single variable domain comprises an amino acid sequence as set forth in SEQ ID NO: 17, or an amino acid sequence having at least 85%, at least 90%, at least 95% or higher sequence identity to SEQ ID NO: 17;
or
the humanized antibody or antigen-binding fragment thereof comprises an amino acid sequence as set forth in SEQ ID NO: 12, 14 or 16 or an amino acid sequence having at least 85%, at least 90%, at least 95% or higher sequence identity to SEQ ID NO: 12, 14 or 16; or
the humanized antibody or antigen-binding fragment thereof comprises the amino acid sequence as set forth in SEQ ID NO: 18 or an amino acid sequence having at least 85%, at least 90%, at least 95% or higher sequence identity to SEQ ID NO: 18.

10. (canceled)

11. (canceled)

12. A pharmaceutical composition comprising the antibody or antigen-binding fragment thereof according to claim 1 and a pharmaceutically acceptable carrier.

13. A pharmaceutical combination comprising the antibody or antigen-binding fragment thereof according to claim 1 and an anti-PD-L1 antibody or an antigen-binding fragment thereof.

14. The pharmaceutical combination according to claim 13, wherein the anti-PD-L1 antibody or antigen-binding fragment thereof specifically recognizes and binds to PD-L1, wherein the anti-PD-L1 antibody or antigen-binding fragment thereof comprises an immunoglobulin single variable domain;
preferably, the immunoglobulin single variable domain comprises:
a CDR1 sequence as set forth in SEQ ID NO: 20,
a CDR2 sequence as set forth in SEQ ID NO: 21, and
a CDR3 sequence as set forth in SEQ ID NO: 22;
more preferably, the immunoglobulin single variable domain comprises: 1) an amino acid sequence as set forth in SEQ ID NO: 23; or 2) an amino acid sequence having at least 85%, at least 90%, at least 95% or higher sequence identity to SEQ ID NO: 23.

15. The pharmaceutical combination according to claim 14, wherein the anti-PD-L1 antibody or antigen-binding fragment thereof further comprises an Fc fragment of a human IgG1;
preferably, the PD-L1 antibody or antigen-binding fragment thereof comprises an amino acid sequence as set forth in SEQ ID NO: 24 or an amino acid sequence having at least 85%, at least 90%, at least 95% or higher sequence to SEQ ID NO: 24.

16. The pharmaceutical combination according to claim 13, which is a pharmaceutical composition or a kit.

17. A method for treating a cancer in a subject comprising administering to the subject a therapeutically effective amount of the antibody or antigen-binding fragment thereof according to claim 1, a pharmaceutical composition comprising the antibody or antigen-binding fragment thereof and a pharmaceutically acceptable carrier, or a pharmaceutical combination comprising the antibody or antigen-binding fragment thereof and an anti-PD-L1 antibody or an antigen-binding fragment thereof;
preferably, the cancer is a hematological tumor or a solid tumor.

18. The method according to claim 17, wherein
the solid tumor comprises squamous cell carcinoma, adenocarcinoma, basal cell carcinoma, renal cell carcinoma, breast ductal carcinoma, soft tissue sarcoma, osteosarcoma, melanoma, small cell lung cancer, non-small cell lung cancer, lung adenocarcinoma, peritoneal cancer, hepatocellular carcinoma, gastrointestinal cancer, gastric cancer, pancreatic cancer, neuroendocrine carcinoma, glioblastoma, cervical cancer, ovarian cancer, bladder cancer, brain cancer, hepatoma, breast cancer, colon cancer, colorectal cancer, endometrial cancer or uterine cancer, esophageal cancer, salivary gland cancer, kidney cancer, liver cancer, prostate cancer, vulvar cancer, thyroid cancer or head and neck cancer;
the hematological tumor comprises leukemia, lymphoma, myeloma, acute myeloid leukemia, chronic myeloid leukemia, acute lymphoblastic leukemia, chronic lymphocytic leukemia, hairy cell leukemia, Hodgkin's lymphoma, non-Hodgkin's lymphoma, or multiple myeloma.

19. The method according to claim 17, further comprising administering one or more therapeutic agents selected from the group consisting of a chemotherapeutic agent, a radioisotope, an immune checkpoint inhibitor, and a tumor antigen targeting drug.

20. An isolated nucleic acid molecule encoding the antibody or antigen-binding fragment thereof according to claim 1.

21. An expression vector or a host cell comprising the nucleic acid molecule according to claim 20.

22. (canceled)

23. A method for producing the antibody or antigen-binding fragment thereof according to claim 1, comprising:
a) culturing a host cell under suitable conditions to express the antibody or antigen-binding fragment thereof, wherein the host cell comprises an isolated nucleic acid molecule encoding the antibody or antigen-binding fragment thereof; and
b) isolating the antibody or antigen-binding fragment thereof from the host cell or a culture thereof.
